(19) United States
(12) Patent Application Publication
(10) Pub. No.: US 2010/0326218 A1
(43) Pub. Date: Dec. 30, 2010

(54) IDENTIFYING A SUBJECT WITH AN INCREASED RISK OF INVASIVE MOLD INFECTION

(76) Inventors: Michael Boeckh, Seattle, WA (US); Jason W. Chien, Seattle, WA (US); Kieren Marr, Baltimore, MD (US); Pierre-Yves Bochud, Pully (CH)

Correspondence Address: MYERS BIGEL SIBLEY & SAJOVEC, PO BOX 37428, RALEIGH, NC, 27627, US

(21) Appl. No.: 12/680,410

(22) PCT Filed: Sep. 26, 2008
(86) PCT No.: PCT/US08/11156

§ 371 Date: Sep. 3, 2010

Related U.S. Application Data
(60) Provisional application No. 60/975,678, filed on Sep. 27, 2007.

(51) Int. Cl. G01N 33/00

(52) U.S. Cl. 73/865.8

(57) ABSTRACT

The present invention provides a method of identifying a subject having a haplotype in a toll-like receptor 4 gene of the subject that is associated with an increased risk of invasive mold infection in a recipient of a transplant from the subject, comprising genotyping the subject for the presence of particular single nucleotide polymorphisms or haplotypes in the toll-like receptor 4 gene of the subject, wherein the detection of said single nucleotide polymorphism(s) and/or haplotype(s) identifies the subject as having a single nucleotide polymorphism and/or haplotype associated with an increased risk of invasive mold infection in a recipient of a transplant from the subject. Also provided herein are methods of identifying an immunocompromised subject as having an increased risk of invasive mold infection and/or increased risk of mortality and/or increased risk of an impaired or altered immune response, by detecting the single nucleotide polymorphisms or haplotypes in the toll-like receptor 4 gene of this invention in the subject.

### STATEMENT OF PRIORITY

This application claims the benefit, under 35 U.S.C. §119(e), of U.S. Provisional Application No. 60/975,678, filed Sep. 27, 2007, the entire content of which is incorporated by reference herein in its entirety.

### GOVERNMENT SUPPORT

Aspects of the present invention were made with the support of federal grant numbers CA18029, CA15704, Al054523, Al051468, Al33484, HL87690 and HL69860 from the National Institutes of Health and US Public Health Service Resource Grant RR04655 from the National Center for Research Resources. The United States Government has certain rights to this invention.

### FIELD OF THE INVENTION

The present invention provides methods and compositions directed to identification of genetic markers associated with impaired immune responses, including increased risk of invasive mold infection in a subject (e.g., an immunocompromised subject) and/or in a recipient of a transplant from a subject carrying the genetic marker(s) described herein.

### BACKGROUND OF THE INVENTION

Over the last 20 years, invasive aspergillosis (IA) has become increasingly frequent among allogenic hematopoietic cell transplant (HCT) patients, with an incidence rate of up to 12%.¹ Despite the availability of new azoles and echinochandins, the outcome remains poor, with a 1-year mortality of 50-80%, making IA one of the leading infection-related causes of death among allogenic HCT patients.^{1,2} Identification of patients with high risk for infection would facilitate development of effective prevention strategies.

Toll-like receptors (TLRs) are transmembrane proteins on the surface of immune cells that detect conserved molecular motifs known as “microbe associated molecular patterns” from a variety of organisms. They interact with several adaptor proteins to activate transcription factors, leading to the production of inflammatory cytokines and the activation of the adaptive immunity.^{3,4} Several studies using TLR deficient mice and/or cellular complementation systems with human or murine TLR plasmids showed that Aspergillus species activate innate immune cells and cytokine production through both TLR2 and TLR4.⁵-10

Common polymorphisms in TLR genes have been associated with susceptibility to several infections.¹¹ Mutations in TLR adaptor molecules (IRAK4, IKKγ and IκBα) cause rare inherited immune deficiencies, further demonstrating the critical importance of genetic variability in the TLR signaling pathway.¹¹

The present invention overcomes previous shortcomings in the art by identifying genetic markers in the TLR4 gene that are associated with an increased risk of invasive mold infection in a recipient of transplanted cells and that are also associated with a subject's ability to elicit an effective immune response in certain immunocompromised and/or disease states (e.g., cancer).

### SUMMARY OF THE INVENTION

Thus, in one aspect, the present invention provides a method of identifying a subject having a haplotype in a toll-like receptor 4 gene of the subject that is associated with an increased risk of invasive mold infection in a recipient of a transplant from the subject, comprising genotyping the subject for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the subject, wherein the detection of said S4 haplotype identifies the subject as having a haplotype associated with an increased risk of invasive mold infection in a recipient of a transplant from the subject.

In additional embodiments, the present invention provides a method of screening a transplant donor for a haplotype in a toll-like receptor 4 gene of the donor that is associated with an increased risk of invasive mold infection in a recipient of a transplant from the donor, comprising genotyping the donor for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the donor, wherein the detection of said S4 haplotype identifies the donor as having a haplotype associated with an increased risk of invasive mold infection in a recipient of a transplant from the donor.

The present invention further provides a method of identifying a subject having a haplotype in a toll-like receptor 4 gene of the subject that is associated with an increased risk of invasive mold infection in a recipient of a transplant from the subject, comprising genotyping the subject for the presence of an S2 haplotype comprising a G allele of the single nucleotide polymorphism rs10759931 (−2604) and a G allele of the single nucleotide polymorphism rs7873784 (+12186) in the toll-like receptor 4 gene of the subject, wherein the detection of said S2 haplotype identifies the subject as having a haplotype associated with an increased risk of invasive mold infection in a recipient of a transplant from the subject.

In additional embodiments, the present invention provides a method of screening a transplant donor for a haplotype in a toll-like receptor 4 gene of the donor that is associated with an increased risk of invasive mold infection in a recipient of a transplant from the donor, comprising genotyping the donor for the presence of an S2 haplotype comprising a G allele of the single nucleotide polymorphism rs10759931 (−2604) and a G allele of the single nucleotide polymorphism rs7873784 (+12186) in the toll-like receptor 4 gene of the donor, wherein the detection of said S2 haplotype identifies the donor as having a haplotype associated with an increased risk of invasive mold infection in a recipient of a transplant from the donor.

Further provided herein is a method of identifying a subject having a haplotype in a toll-like receptor 4 gene of the subject that is associated with an increased risk of invasive mold infection in a recipient of a transplant from the subject, comprising genotyping the subject for the presence of a haplotype in the toll-like receptor gene of the subject selected from the group consisting of: a) H5; b) H6; and c) H8, wherein the detection of said haplotype identifies the subject as having a haplotype associated with an increased risk of invasive mold infection in a recipient of a transplant from the subject.

Further aspects of the present invention include a method of screening a transplant donor for a haplotype in a toll-like receptor 4 gene of the donor that is associated with an increased risk of invasive mold infection in a recipient of a transplant from the donor, comprising genotyping the donor for the presence of a haplotype in the toll-like receptor gene of the donor selected from the group consisting of: a) H5; b) H6; and c) H8, wherein the detection of said haplotype identifies the donor as having a haplotype associated with an increased risk of invasive mold infection in a recipient of a transplant from the donor.

The present invention additionally provides a method of identifying a subject having a single nucleotide polymorphism allele or a combination of single nucleotide polymorphism alleles in a toll-like receptor 4 gene of the subject that is associated with an increased risk of invasive mold infection in a recipient of a transplant from the subject, comprising genotyping the subject for the presence of one or more than one single nucleotide polymorphism allele in the toll-like receptor gene of the subject selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
   wherein the detection of said single nucleotide polymorphism allele or combination of single nucleotide polymorphism alleles identifies the subject as having a single nucleotide polymorphism allele or a combination of single nucleotide polymorphism alleles associated with an increased risk of invasive mold infection in a recipient of a transplant from the subject.

Furthermore, the present invention provides a method of screening a transplant donor for a single nucleotide polymorphism allele or a combination of single nucleotide polymorphism alleles in a toll-like receptor 4 gene of the donor that is associated with an increased risk of invasive mold infection in a recipient of a transplant from the donor, comprising genotyping the donor for the presence of one or more than one single nucleotide polymorphism allele in the toll-like receptor gene of the donor selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
   wherein the detection of said single nucleotide polymorphism allele or combination of single nucleotide polymorphism alleles identifies the donor as having a single nucleotide polymorphism allele or a combination of single nucleotide polymorphism alleles associated with an increased risk of invasive mold infection in a recipient of a transplant from the donor.

Also provided herein is a method of identifying a transplant recipient as having an increased risk of invasive mold infection following transplantation, comprising detecting in the transplant recipient a haplotype of a toll-like receptor 4 gene that is associated with an increased risk of invasive mold infection in a transplant recipient, comprising genotyping the transplant recipient for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the transplant recipient, wherein the detection of said S4 haplotype identifies the transplant recipient as having a haplotype associated with an increased risk of invasive mold infection following transplantation and thereby identifies the transplant recipient as having an increased risk of invasive mold infection following transplantation.

Additional aspects of the present invention include a method of identifying a transplant recipient as having an increased risk of invasive mold infection following transplantation, comprising detecting in the transplant recipient a haplotype in a toll-like receptor 4 gene of the transplant recipient that is associated with an increased risk of invasive mold infection in a recipient of a transplant, comprising genotyping the transplant recipient for the presence of a haplotype selected from the group consisting of: a) H5; b) H6; and c) H8, wherein the detection of said haplotype identifies the transplant recipient as having a haplotype associated with an increased risk of invasive mold infection in a recipient of a transplant, thereby identifying the transplant recipient as having an increased risk of invasive mold infection following transplantation.

The present invention also provides a method of identifying a transplant recipient as having an increased risk of invasive mold infection following transplantation, comprising detecting in the transplant recipient a single nucleotide polymorphism allele or a combination of single nucleotide polymorphism alleles in a toll-like receptor 4 gene of the transplant recipient that is associated with an increased risk of invasive mold infection in a recipient of a transplant, comprising genotyping the transplant recipient for the presence of one or more than one single nucleotide polymorphism allele in the toll-like receptor gene of the transplant recipient selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
   wherein the detection of said single nucleotide polymorphism allele or combination of single nucleotide polymorphism alleles identifies the transplant recipient as having a single nucleotide polymorphism allele or a combination of single nucleotide polymorphism alleles associated with an increased risk of invasive mold infection in a recipient of a transplant, thereby identifying the transplant recipient as having an increased risk of invasive mold infection following transplantation.

Further aspects of the present invention include a method of identifying an immunocompromised subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising detecting in the subject a haplotype of a toll-like receptor 4 gene that is associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising genotyping the subject for the presence of an S4 haplotype in the toll-like receptor 4 gene of the subject, wherein the detection of said S4 haplotype identifies the subject as having a haplotype associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen and thereby identifies the subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen.

Further aspects of the present invention include a method of identifying an immunocompromised subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising detecting in the subject a haplotype of a toll-like receptor 4 gene that is associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising genotyping the subject for the presence of an S2 haplotype in the toll-like receptor 4 gene of the subject, wherein the detection of said S2 haplotype identifies the subject as having a haplotype associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen and thereby identifies the subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen.

Also provided herein is a method of identifying a transplant recipient as having an increased risk of invasive mold infection following transplantation, comprising detecting in the transplant recipient a haplotype of a toll-like receptor 4 gene that is associated with an increased risk of invasive mold infection in a transplant recipient, comprising genotyping the transplant recipient for the presence of an S2 haplotype comprising a G allele of the single nucleotide polymorphism rs10759931 (−2604) and a G allele of the single nucleotide polymorphism rs7873784 (+12186) in the toll-like receptor 4 gene of the transplant recipient, wherein the detection of said S2 haplotype identifies the transplant recipient as having a haplotype associated with an increased risk of invasive mold infection following transplantation and thereby identifies the transplant recipient as having an increased risk of invasive mold infection following transplantation.

Further provided herein is a method of identifying a immunocompromised subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising detecting in the subject a haplotype in a toll-like receptor 4 gene of the subject that is associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising genotyping the subject for the presence of a haplotype selected from the group consisting of: a) H5; b) H6; and c) H8, wherein the detection of said haplotype identifies the subject as having a haplotype associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, thereby identifying the subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen.

Further aspects of this invention include a method of identifying an immunocompromised subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising detecting in the subject a single nucleotide polymorphism allele or a combination of single nucleotide polymorphism alleles in a toll-like receptor 4 gene of the subject that is associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising genotyping the subject for the presence of one or more than one single nucleotide polymorphism allele in the toll-like receptor gene of the subject selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
   wherein the detection of said single nucleotide polymorphism allele or combination of single nucleotide polymorphism alleles identifies the subject as having a single nucleotide polymorphism allele or a combination of single nucleotide polymorphism alleles associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, thereby identifying the subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen.

Yet further aspects of the present invention include a method of screening for increased risk of invasive mold infection or increased mortality in an immunocompromised subject, wherein the presence of a haplotype in the toll-like receptor 4 (TLR4) gene of the subject selected from the group consisting of: a) an S4 haplotype; b) an H5 haplotype; c) an H6 haplotype; and d) an H8 haplotype, indicates said subject is at increased risk of an invasive mold infection and/or increased likelihood of mortality, comprising detecting the presence or absence of said haplotype in a biological sample of said subject.

Also provided herein is a method of identifying a subject of this invention (e.g., a transplant donor, a transplant recipient and/or an immunocompromised or “high risk” subject) as having an increased risk of invasive mold infection, comprising genotyping the subject for the presence of a G allele of the single nucleotide polymorphism rs4986790 (1063) and a T allele of the single nucleotide polymorphism rs4986791 (1363), wherein the detection of said alleles identifies the subject as having an increased risk of invasive mold infection.

The present invention further provides the use of a means of detecting a haplotype of a toll-like receptor 4 gene, wherein said haplotype is selected from the group consisting of: a) an S4 haplotype; b) an H5 haplotype; c) an H6 haplotype; and d) an H8 haplotype, in a biological sample of an immunocompromised subject, in determining if said subject is at increased risk of an invasive mold infection or mortality.

In additional aspects of this invention, a method is provided of identifying a transplant recipient as having an increased risk of invasive mold infection following transplantation, comprising detecting in a donor tissue and/or cell in the transplant recipient a haplotype of a toll-like receptor 4 gene that is associated with an increased risk of invasive mold infection in a transplant recipient, comprising genotyping the donor tissue and/or cell in the transplant recipient for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the transplant recipient, wherein the detection of said S4 haplotype in the donor tissue and/or cell in the transplant recipient identifies the transplant recipient as having an increased risk of invasive mold infection following transplantation.

Furthermore, a method is provided of identifying a transplant recipient as having an increased risk of invasive mold infection following transplantation, comprising detecting in a donor tissue and/or cell in the transplant recipient a haplotype in a toll-like receptor 4 gene that is associated with an increased risk of invasive mold infection in a recipient of a transplant, comprising genotyping the donor tissue and/or cell in the transplant recipient for the presence of a haplotype selected from the group consisting of: a) H5; b) H6; and c) H8, wherein the detection of said haplotype in the donor tissue and/or cell identifies the transplant recipient as having an increased risk of invasive mold infection following transplantation.

In addition, a method is provided of identifying a transplant recipient as having an increased risk of invasive mold infection following transplantation, comprising detecting in a donor tissue and/or cell in the transplant recipient a single nucleotide polymorphism allele or a combination of single nucleotide polymorphism alleles in a toll-like receptor 4 gene that is associated with an increased risk of invasive mold infection in a recipient of a transplant, comprising genotyping the donor tissue and/or cell of the transplant recipient for the presence of one or more than one single nucleotide polymorphism allele in the toll-like receptor gene selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
   wherein the detection in the donor tissue and/or cell of said single nucleotide polymorphism allele or combination of single nucleotide polymorphism alleles identifies the transplant recipient as having an increased risk of invasive mold infection following transplantation.

Also provided herein is a method of identifying a transplant recipient as having an increased risk of invasive mold infection following transplantation, comprising genotyping a donor tissue and/or cell in the transplant recipient for the presence of a G allele of the single nucleotide polymorphism rs4986790 (1063) and a T allele of the single nucleotide polymorphism rs4986791 (1363), wherein the detection of said alleles in a donor tissue and/or cell identifies the transplant recipient as having an increased risk of invasive mold infection following transplantation.

### BRIEF DESCRIPTION OF THE DRAWINGS

FIGS. 1A-E. Cumulative Incidence of Invasive Aspergillosis and Death after Hematopoietic Stem Cell Transplantation According to Donor TLR4 Haplotypes and CMV Serostatus. The end point was the time to diagnosis of proven or probable invasive aspergillosis in 242 eligible patients, with censoring at the date of last follow-up, and considering relapsed malignancy, subsequent HCT and death as competing risks. P are P values for the univariate Cox regression model (Panels A, B, D), or the log rank text (Panel C, no event in the reference group). Haplotypes H6 and H8 were associated with an increased incidence of invasive aspergillosis during the first 6 months after transplantation and were grouped together (Panel A). Seven cases in the haplotype H6/H8 group had both donor H6/H8 and H5. Haplotypes H5, H6 and H8 could be grouped under a unique 2 loci haplotype, characterized by a G at position −2604 and a G at position +12186 (Haplotype S2, Panel B). S2 comprised also a few donor haplotypes other than H5, H6 or H8 (e.g. frequency <2%), so that the total number of donors with S2 is slightly bigger than the addition of donors with H5, H6 and H8. Since both CMV serostatus and TLR4 haplotypes can be assessed before HCT, cumulative incidence was stratified by CMV serostatus (D−R−, denoted CMV−) versus the other patients (D−R+, D+R+ and D+R−, denoted CMV+) and the presence or the absence of haplotype S2 in the donor (denoted S2+ versus S2−, Panels C and D). Haplotypes were inferred for Caucasian donors and recipients together using the S.A.G.E. software (Panel E). TLR4 1063 A/G and 1363 C/T are in strong LD and can be used interchangeably within the 6 loci haplotype. Based on reference sequences having GenBank® Accession Nos. NM_{—}138554, NT_{—}008470 and NP_{—}612564. Rare haplotypes (<2%) are not represented.

FIGS. 2A-D. Cumulative Incidence of Invasive Aspergillosis and Death after Hematopoietic Stem Cell Transplantation According to Donor TLR4 Haplotypes and CMV Serostatus in the Discovery Study. The end point was the time to diagnosis of proven or probable invasive aspergillosis in 242 eligible patients, with censoring at the date of last follow-up, and considering relapsed malignancy, subsequent HCT and death as competing risks. P are P values for the log rank test. Since both CMV serostatus and TLR4 haplotypes can be assessed before HCT, cumulative incidences were stratified in different groups by CMV serostatus (D−R−, denoted CMV−) versus the other patients (D−R+, D+R+ and D+R−, denoted “CMV+”) and the presence or the absence of donor S4 (denoted “S4+” and “S4−”) (Panels A-C). Haplotypes were inferred for Caucasian donors and recipients together using the S.A.G.E. software (Panel D). TLR4 1063 A/G and 1363 C/T are in strong LD and can be used interchangeably within the 6 loci and 3 loci haplotypes. Based on reference sequences having GenBank® Accession Nos. NM_{—}138554, NT_{—}008470 and NP_{—}612564. Rare haplotypes (<2%) are not represented.

### DETAILED DESCRIPTION OF THE INVENTION

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

In some embodiments, the present invention is based on the unexpected discovery of the association between certain genetic markers in the toll-like receptor 4 (TLR4) gene and increased incidence of, as well as the identification of increased risk of, invasive mold infection and/or death in a transplant recipient. In some embodiments, the genetic marker(s) of this invention can be present in cells of a transplant donor and/or present in cells of a transplant recipient.

Further aspects of the present invention are based on the unexpected discovery of the association between certain genetic markers in the TLR4 gene of an immunocompromised subject and increased risk of invasive mold infection, increased risk of invasive mold infection following chemotherapy and/or radiation treatment, increased risk of a poor prognosis (e.g., in a transplant recipient, a cancer patient and/or a critically ill subject) and/or decreased likelihood of eliciting an effective immune response (i.e., having an impaired or altered immune response) to an immunogen (e.g., a vaccine, a pathogen such as a mold).

Thus, in one aspect, the present invention provides a method of identifying a subject having a haplotype in a toll-like receptor 4 gene of the subject that is associated with an increased risk of invasive mold infection in a recipient of a transplant from the subject, comprising genotyping the subject for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the subject, wherein the detection of said S4 haplotype identifies the subject as having a haplotype associated with an increased risk of invasive mold infection in a recipient of a transplant from the subject.

In additional embodiments, the present invention provides a method of screening a transplant donor for a haplotype in a toll-like receptor 4 gene of the donor that is associated with an increased risk of invasive mold infection in a recipient of a transplant from the donor, comprising genotyping the donor for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the donor, wherein the detection of said haplotype identifies the donor as having a haplotype associated with an increased risk of invasive mold infection in a recipient of a transplant from the donor.

In a further embodiment, the present invention provides a method of identifying a subject having a haplotype in a toll-like receptor 4 gene of the subject that is associated with an increased risk of invasive mold infection in a recipient of a transplant from the subject, comprising genotyping the subject for the presence of an S2 haplotype comprising a G allele of the single nucleotide polymorphism rs10759931 and a G allele of the single nucleotide polymorphism rs7873784 in the toll-like receptor 4 gene of the subject, wherein the detection of said S2 haplotype identifies the subject as having a haplotype associated with an increased risk of invasive mold infection in a recipient of a transplant from the subject.

Also provided herein is a method of screening a transplant donor for a haplotype in a toll-like receptor 4 gene of the donor that is associated with an increased risk of invasive mold infection in a recipient of a transplant from the donor, comprising genotyping the donor for the presence of an S2 haplotype comprising a G allele of the single nucleotide polymorphism rs10759931 and a G allele of the single nucleotide polymorphism rs7873784 in the toll-like receptor 4 gene of the donor, wherein the detection of said haplotype identifies the donor as having a haplotype associated with an increased risk of invasive mold infection in a recipient of a transplant from the donor.

Further embodiments of the present invention include a method of identifying a subject having a haplotype in a toll-like receptor 4 gene of the subject that is associated with an increased risk of invasive mold infection in a recipient of a transplant from the subject, comprising genotyping the subject for the presence of a haplotype in the toll-like receptor gene of the subject selected from the group consisting of: a) H5; b) H6; and c) H8, wherein the detection of said haplotype identifies the subject as having a haplotype associated with an increased risk of invasive mold infection in a recipient of a transplant from the subject.

Furthermore, the present invention provides a method of screening a transplant donor for a haplotype in a toll-like receptor 4 gene of the donor that is associated with an increased risk of invasive mold infection in a recipient of a transplant from the donor, comprising genotyping the donor for the presence of a haplotype in the toll-like receptor gene of the donor selected from the group consisting of: a) H5; b) H6; and c) H8, wherein the detection of said haplotype identifies the donor as having a haplotype associated with an increased risk of invasive mold infection in a recipient of a transplant from the donor.

The H5 haplotype of this invention comprises, consists essentially of and/or consists of an A allele of the single nucleotide polymorphism rs2770150 (−3612), a G allele of the single nucleotide polymorphism rs10759931 (−2604), a C allele of the single nucleotide polymorphism rs10759932 (−1607), an A allele of the single nucleotide polymorphism rs4986790 (1063), a C allele of the single nucleotide polymorphism rs4986791 (1363), a G allele of the single nucleotide polymorphism rs11536889 (+11381) and a G allele of the single nucleotide polymorphism rs7873784 (+12186).

The H6 haplotype of this invention comprises, consists essentially of and/or consists of an A allele of the single nucleotide polymorphism rs2770150 (−3612), a G allele of the single nucleotide polymorphism rs10759931 (−2604), a T allele of the single nucleotide polymorphism rs10759932 (−1607), a G allele of the single nucleotide polymorphism rs4986790 (1063), a T allele of the single nucleotide polymorphism rs4986791 (1363), a G allele of the single nucleotide polymorphism rs11536889 (+11381) and a G allele of the single nucleotide polymorphism rs7873784 (+12186).

The H8 haplotype of this invention comprises, consists essentially of and/or consists of an A allele of the single nucleotide polymorphism rs2770150 (−3612), a G allele of the single nucleotide polymorphism rs10759931 (−2604), a T allele of the single nucleotide polymorphism rs10759932 (−1607), an A allele of the single nucleotide polymorphism rs4986790 (1063), a C allele of the single nucleotide polymorphism rs4986791 (1363), a G allele of the single nucleotide polymorphism rs11536889 (+11381) and a G allele of the single nucleotide polymorphism rs7873784 (+12186).

Additionally provided herein is a method of identifying a subject having a single nucleotide polymorphism allele or a combination of single nucleotide polymorphism alleles in a toll-like receptor 4 gene of the subject that is associated with an increased risk of invasive mold infection in a recipient of a transplant from the subject, comprising genotyping the subject for the presence of one or more than one single nucleotide polymorphism allele in the toll-like receptor gene of the subject selected from the group consisting of: a) a G allele of the single nucleotide polymorphism rs10759931 (−2604); b) a G allele of the single nucleotide polymorphism rs7873784 (+12186); c) an A allele of the single nucleotide polymorphism rs4986790 (1063); d) a G allele of the single nucleotide polymorphism rs4986790 (1063); e) a T allele of the single nucleotide polymorphism rs4986791 (1363); f) a C allele of the single nucleotide polymorphism rs4986791 (1363); g) an A allele of the single nucleotide polymorphism rs2770150 (−3612); h) a C allele of the single nucleotide polymorphism rs10759932 (−1607); i) a T allele of the single nucleotide polymorphism rs10759932 (−1607); j) a G allele of the single nucleotide polymorphism rs11536889 (+11381); and k) any combination of (a)-(j) above, wherein the detection of said single nucleotide polymorphism allele or combination of single nucleotide polymorphism alleles identifies the subject as having a single nucleotide polymorphism allele or a combination of single nucleotide polymorphism alleles associated with an increased risk of invasive mold infection in a recipient of a transplant from the subject.

Furthermore, the present invention provides a method of screening a transplant donor for a single nucleotide polymorphism allele or a combination of single nucleotide polymorphism alleles in a toll-like receptor 4 gene of the donor that is associated with an increased risk of invasive mold infection in a recipient of a transplant from the donor, comprising genotyping the donor for the presence of one or more than one single nucleotide polymorphism allele in the toll-like receptor gene of the donor selected from the group consisting of: a) a G allele of the single nucleotide polymorphism rs10759931 (−2604); b) a G allele of the single nucleotide polymorphism rs7873784 (+12186); c) an A allele of the single nucleotide polymorphism rs4986790 (1063); d) a G allele of the single nucleotide polymorphism rs4986790 (1063); e) a T allele of the single nucleotide polymorphism rs4986791 (1363); f) a C allele of the single nucleotide polymorphism rs4986791 (1363); g) an A allele of the single nucleotide polymorphism rs2770150 (−3612); h) a C allele of the single nucleotide polymorphism rs10759932 (−1607); i) a T allele of the single nucleotide polymorphism rs10759932 (−1607); j) a G allele of the single nucleotide polymorphism rs11536889 (+11381); and k) any combination of (a)-(j) above, wherein the detection of said single nucleotide polymorphism allele or combination of single nucleotide polymorphism alleles identifies the donor as having a single nucleotide polymorphism allele or a combination of single nucleotide polymorphism alleles associated with an increased risk of invasive mold infection in a recipient of a transplant from the donor.

Also provided herein is a method of identifying a transplant recipient as having an increased risk of invasive mold infection following transplantation, comprising detecting in the transplant recipient a haplotype of a toll-like receptor 4 gene that is associated with an increased risk of invasive mold infection in a transplant recipient, comprising genotyping the transplant recipient for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the transplant recipient, wherein the detection of said S4 haplotype identifies the transplant recipient as having a haplotype associated with an increased risk of invasive mold infection following transplantation and thereby identifies the transplant recipient as having an increased risk of invasive mold infection following transplantation.

A method is also provided herein of identifying a transplant recipient as having an increased risk of invasive mold infection following transplantation, comprising detecting in the transplant recipient a haplotype of a toll-like receptor 4 gene that is associated with an increased risk of invasive mold infection in a transplant recipient, comprising genotyping the transplant recipient for the presence of an S2 haplotype comprising a G allele of the single nucleotide polymorphism rs10759931 and a G allele of the single nucleotide polymorphism rs7873784 in the toll-like receptor 4 gene of the transplant recipient, wherein the detection of said S2 haplotype identifies the transplant recipient as having a haplotype associated with an increased risk of invasive mold infection following transplantation and thereby identifies the transplant recipient as having an increased risk of invasive mold infection following transplantation.

Other embodiments of this invention include a method of identifying a transplant recipient as having an increased risk of invasive mold infection following transplantation, comprising detecting in the transplant recipient a haplotype in a toll-like receptor 4 gene of the transplant recipient that is associated with an increased risk of invasive mold infection in a recipient of a transplant, comprising genotyping the transplant recipient for the presence of a haplotype selected from the group consisting of: a) H5; b) H6; and c) H8, wherein the detection of said haplotype identifies the transplant recipient as having a haplotype associated with an increased risk of invasive mold infection in a recipient of a transplant, thereby identifying the transplant recipient as having an increased risk of invasive mold infection following transplantation.

Further provided herein is a method of identifying a transplant recipient as having an increased risk of invasive mold infection following transplantation, comprising detecting in the transplant recipient a single nucleotide polymorphism allele or a combination of single nucleotide polymorphism alleles in a toll-like receptor 4 gene of the transplant recipient that is associated with an increased risk of invasive mold infection in a recipient of a transplant, comprising genotyping the transplant recipient for the presence of one or more than one single nucleotide polymorphism allele in the toll-like receptor gene of the transplant recipient selected from the group consisting of: a) a G allele of the single nucleotide polymorphism rs10759931 (−2604); b) a G allele of the single nucleotide polymorphism rs7873784 (+12186); c) an A allele of the single nucleotide polymorphism rs4986790 (1063); d) a G allele of the single nucleotide polymorphism rs4986790 (1063); e) a T allele of the single nucleotide polymorphism rs4986791 (1363); f) a C allele of the single nucleotide polymorphism rs4986791 (1363); g) an A allele of the single nucleotide polymorphism rs2770150 (−3612); h) a C allele of the single nucleotide polymorphism rs10759932 (−1607); i) a T allele of the single nucleotide polymorphism rs10759932 (−1607); j) a G allele of the single nucleotide polymorphism rs11536889 (+11381); and k) any combination of (a)-(j) above, wherein the detection of said single nucleotide polymorphism allele or combination of single nucleotide polymorphism alleles identifies the transplant recipient as having a single nucleotide polymorphism allele or a combination of single nucleotide polymorphism alleles associated with an increased risk of invasive mold infection in a recipient of a transplant, thereby identifying the transplant recipient as having an increased risk of invasive mold infection following transplantation.

In further embodiments, the present invention provides a method of identifying an immunocompromised subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising detecting in the subject a haplotype of a toll-like receptor 4 gene that is associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising genotyping the subject for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the transplant recipient, wherein the detection of said S4 haplotype identifies the subject as having a haplotype associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen and thereby identifies the subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen.

In yet further embodiments, the present invention provides a method of identifying an immunocompromised subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising detecting in the subject a haplotype of a toll-like receptor 4 gene that is associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising genotyping the subject for the presence of an S2 haplotype comprising a G allele of the single nucleotide polymorphism rs10759931 and a G allele of the single nucleotide polymorphism rs7873784 in the toll-like receptor 4 gene of the subject, wherein the detection of said S2 haplotype identifies the subject as having a haplotype associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen and thereby identifies the subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen.

Further provided herein is a method of identifying a immunocompromised subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising detecting in the subject a haplotype in a toll-like receptor 4 gene of the subject that is associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising genotyping the subject for the presence of a haplotype selected from the group consisting of: a) H5; b) H6; and c) H8, wherein the detection of said haplotype identifies the subject as having a haplotype associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, thereby identifying the subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen.

Further aspects of this invention include a method of identifying an immunocompromised subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising detecting in the subject a single nucleotide polymorphism allele or a combination of single nucleotide polymorphism alleles in a toll-like receptor 4 gene of the subject that is associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, comprising genotyping the subject for the presence of one or more than one single nucleotide polymorphism allele in the toll-like receptor gene of the subject selected from the group consisting of: a) a G allele of the single nucleotide polymorphism rs10759931 (−2604); b) a G allele of the single nucleotide polymorphism rs7873784 (+12186); c) an A allele of the single nucleotide polymorphism rs4986790 (1063); d) a G allele of the single nucleotide polymorphism rs4986790 (1063); e) a T allele of the single nucleotide polymorphism rs4986791 (1363); f) a C allele of the single nucleotide polymorphism rs4986791 (1363); g) an A allele of the single nucleotide polymorphism rs2770150 (−3612); h) a C allele of the single nucleotide polymorphism rs10759932 (−1607); i) a T allele of the single nucleotide polymorphism rs10759932 (−1607); j) a G allele of the single nucleotide polymorphism rs11536889 (+11381); and k) any combination of (a)-(j) above, wherein the detection of said single nucleotide polymorphism allele or combination of single nucleotide polymorphism alleles identifies the subject as having a single nucleotide polymorphism allele or a combination of single nucleotide polymorphism alleles associated with 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen, thereby identifying the subject as having 1) an increased risk of invasive mold infection, 2) an increased risk of invasive mold infection following chemotherapy and/or radiation therapy; 3) an increased risk of a poor disease prognosis; and/or a decreased likelihood of eliciting an effective immune response to an immunogen.

Additionally provided herein is a method of identifying an immunocompromised subject and/or other subject of this invention as having an increased risk of invasive mold infection, comprising genotyping the subject for the presence of a G allele of the single nucleotide polymorphism rs4986790 (1063) and a T allele of the single nucleotide polymorphism rs4986791 (1363), wherein the detection of said alleles identifies the subject as having an increased risk of invasive mold infection.

Thus, the methods of this invention can be used, in some embodiments, to screen transplant donors in order to identify those donors whose cells and/or organs may not be suitable for a particular transplant recipient, due to the presence in the donor cells or organ of the markers of this invention that impart increased risks to the transplant recipient.

It is further contemplated that the methods of this invention can be carried out on donor tissue and/or cells after such donor tissue and/or cells has been transplanted into the transplant recipient. Thus, further aspects of this invention include identifying a transplant recipient as having an increased risk of invasive mold infection following transplantation, comprising detecting in donor tissue and/or cells in the transplant recipient a haplotype of a toll-like receptor 4 gene that is associated with an increased risk of invasive mold infection in a transplant recipient. These methods can be carried out by genotyping donor tissue and/or cells from the transplant recipient for the presence of a haplotype of this invention (e.g., S2, S4, H5, H6, H8), as well as for the presence of the alleles described herein (e.g., a) a G allele of the single nucleotide polymorphism rs10759931 (−2604); b) a G allele of the single nucleotide polymorphism rs7873784 (+12186); c) an A allele of the single nucleotide polymorphism rs4986790 (1063); d) a G allele of the single nucleotide polymorphism rs4986790 (1063); e) a T allele of the single nucleotide polymorphism rs4986791 (1363); f) a C allele of the single nucleotide polymorphism rs4986791 (1363); g) an A allele of the single nucleotide polymorphism rs2770150 (−3612); h) a C allele of the single nucleotide polymorphism rs10759932 (−1607); i) a T allele of the single nucleotide polymorphism rs10759932 (−1607); j) a G allele of the single nucleotide polymorphism rs11536889 (+11381); and k) any combination of (a)-(j) above.

The methods of this invention are also useful in identifying donors and/or donor tissues/cells carrying the markers of this invention in situations where transplantation of the donor's cells and/or organs is desirable and/or necessary even in view of the increased risk to the recipient, in order to prepare and/or treat the recipient before, during and/or after transplantation to address the increased risk of invasive mold infection and/or impaired immune response. Such treatments and preparations would be well known to one of skill in the art.

In the methods of this invention directed to increased risk of invasive mold infection, such an infection can be caused by any fungus capable of causing an infection in a subject of this invention, including but not limited to Aspergillus species, Fusarium species, Mucor species, Rhizopus species and any combination thereof.

Thus, in the embodiments of the invention as provided herein, a TLR4 allele of this invention can be a G allele of the single nucleotide polymorphism rs10759931 (−2604); a G allele of the single nucleotide polymorphism rs7873784 (+12186); an A allele of the single nucleotide polymorphism rs4986790 (1063); a G allele of the single nucleotide polymorphism rs4986790 (1063); a T allele of the single nucleotide polymorphism rs4986791 (1363); a C allele of the single nucleotide polymorphism rs4986791 (1363); an A allele of the single nucleotide polymorphism rs2770150 (−3612); a C allele of the single nucleotide polymorphism rs10759932 (−1607); a T allele of the single nucleotide polymorphism rs10759932 (−1607); a G allele of the single nucleotide polymorphism rs11536889 (+11381); as well as any combination of these alleles. Thus, the invention provides a combination of two or more TLR4 alleles, a combination of three or more TLR4 alleles, a combination of four or more TLR4 alleles, a combination of five or more TLR4 alleles, a combination of six or more TLR alleles of this invention, etc.

A subject of this invention can have one copy of a TLR4 allele of this invention and be heterozygous for a particular allele or the subject can have two copies of a TLR4 allele of this invention and be homozygous for a particular allele. A subject of this invention can be heterozygous for the alleles of this invention and/or homozygous for the alleles of this invention in any combination.

A subject of this invention can be any subject that has cells comprising a TLR4 gene. In particular embodiments, the subject can be a human. The human subject can be of either gender and of any ethnic origin, including Caucasian, Negro (e.g., African, African American, African European, etc.), Hispanic and Asian.

A subject of this invention can be a “high risk” subject, which means the subject is immunodeficient, immunosuppressed, immunocompromised and/or is more vulnerable than average (e.g., more vulnerable than a subject who is not immunodeficient, immunosuppressed, immunocompromised or not having a stressful event as described herein) to an invasive mold infection and/or mortality due to various stressful events as described herein.

The terms “immunodeficient,” “immunosuppressed” and “immunocompromised” as used herein are intended to have their art-recognized meaning as describing a subject whose immune system is impaired, defective, weakened and/or functioning abnormally as compared with a healthy or normal subject. An immunodeficient, immunosuppressed or immunocompromised state in a subject can be due to a variety of causes that are well known in the art, including but not limited to, genetic disorders of the immune system, diseases, disorders and/or infections that affect the immune system (e.g., human immunodeficiency virus infection and other viral, parasitic and bacterial infections), autoimmune disorders, drug-induced and/or radiation-induced immunosuppression for transplantation and/or to treat various diseases and disorders, steroid use, chemotherapy, radiation therapy and any other therapies that deplete immune cells, splenectomy, cystic fibrosis, sepsis, cancer, kidney failure, alcoholism, cirrhosis, diabetes, pregnancy, old age, infancy, hypothermia, severe emotional trauma or stress, malnutrition, etc., as would be known in the art.

Thus, a subject of this invention can be, for example, a transplant recipient, a cancer patient, a cancer patient undergoing chemotherapy and/or radiation therapy, a critically ill patient, a patient that has an immunosuppressing disease, a subject taking immunosuppressive medication, a subject with an immunodeficiency due to a genetic defect and any combination thereof.

Furthermore, a subject of this invention can be a “high risk” subject as a result of a stressful event. Nonlimiting examples of a stressful event that would identify a subject as “high risk” include placement in a hospital or other medical facility, an elective surgery, a non-elective surgery, an elective invasive procedure, a non-elective invasive procedure, trauma or injury to the subject (e.g., automobile accident, burn, cold exposure, heat exposure and/or other accidental trauma or injury), emotional and/or psychological stress and/or trauma and/or any disease condition or pathological state that can increase the likelihood of development of an invasive mold infection in the subject and/or increase the likelihood of mortality and/or increased risk of an impaired or altered immune response, as would be well known to one of skill in the art.

Thus, for example, in some embodiments of the invention, the subject can be a perioperative patient, a postoperative patient, a preoperative patient, a periprocedural patient, a postprocedural patient, a preprocedural patient, an intensive care unit patient, a post-intensive care unit patient, a trauma patient, an acutely ill patient, a chronically ill patient and any combination of the above.

In other words, a subject of this invention can be a subject who is about to undergo a surgery and/or invasive procedure, a subject who is preparing to undergo a surgery and/or invasive procedure and/or a subject who is about to undergo and/or is preparing to undergo a medical treatment that can increase the likelihood of the development of an invasive mold infection in the subject and/or increase the likelihood of mortality in the subject and/or increased risk of an impaired or altered immune response. In some embodiments, the subject of this invention can be a subject who has undergone a surgery and/or invasive procedure and/or a subject who has undergone a medical treatment that can increase the likelihood of development-of an invasive mold infection in the subject and/or increase the likelihood of mortality in the subject and/or increase the risk of an impaired or altered immune response. Furthermore, the subject of this invention can be a subject who is about to receive and/or who has received medical treatment that does result and/or could result in placement of the subject in an intensive care unit.

As used herein, “perioperative and periprocedural” mean the period of time extending from when the subject goes into a hospital, clinic, doctor's office or other facility for surgery, a procedure and/or other medical treatment until the time the subject returns home. Accordingly, preoperative and preprocedural means the period of time before the subject goes into a hospital, clinic, doctor's office or other facility for surgery, a procedure and/or other medical treatment and postoperative and postprocedure means the period of time after the subject returns home following the surgery, procedure and/or other medical treatment.

Furthermore, as used herein, “an intensive care unit patient” is a subject who has been admitted to an intensive care unit of a hospital, clinic or other medical facility for any medical condition that warrants intensive care, as would be known by one of skill in the art. A “post-intensive care unit patient” is a subject who had previously been cared for in an intensive care unit of a hospital, clinic or other medical facility and has been discharged from the intensive care unit.

Also as used herein, the term “invasive procedure” means any technique where entry to a body cavity is required or where the normal function of the body is in some way interrupted. An invasive procedure can also be a medical procedure and/or treatment that invades (enters) the body, usually by cutting or puncturing the skin and/or by inserting instruments into the body.

Nonlimiting examples of an invasive procedure of this invention include endoscopy, bronchoscopy, cardiac catheterization, angioplasty, colonoscopy, hemodialysis, blood transfusion, blood donation, plasma donation, leukopheresis and any combination thereof.

In addition, nonlimiting examples of a surgery, operation or surgical procedure of this invention include transplantation of an organ or tissue (e.g., hematopoietic cells, hematopoietic stem cells, kidney, skin graft, bone graft, liver, heart, heart valve, lung, pancreas, islet cells, intestines, cornea, hand, foot, etc.), surgery on an organ or tissue (e.g., heart, lung, stomach, kidneys, eye, uterus, ovaries, intestines, colon, brain, prostate, gall bladder, appendix, joint, etc.), removal of organs, bariatric surgery, laparoscopic surgery, hernia surgery, hemorrhoid surgery, plastic surgery, exploratory surgery, varicose vein surgery, minimally invasive surgery, etc.

It is further contemplated that the methods of this invention can be carried out at any time relative to the event that increases the likelihood of development of an invasive mold infection and/or increases the likelihood of mortality in the subject and/or increases the risk of an impaired or altered immune response. Thus, the methods of this invention can be carried out prior to, during and/or after surgery, an invasive procedure, a trauma or injury and/or a treatment that increases the likelihood of invasive mold infection and/or mortality and/or increases the risk of an impaired or altered immune response. For example, in some embodiments, the methods of this invention can also be carried out prior to, during and/or after a subject is a patient in an intensive care unit.

In further embodiments, the methods of this invention can be carried out on a subject who has developed an invasive mold infection, including a current infection, as well as a past incident of infection from which the subject has recovered. In additional embodiments, the subject can have a relative (e.g., parent, sibling, child, aunt, uncle, grandparent, niece, nephew, cousin, etc.) who has developed an invasive mold infection, which can be a current infection and/or a past incident of infection.

An allele of the TLR4 gene is correlated with the increased risks described herein or correlated with a decreased likelihood of eliciting an effective immune response to an immunogen by identifying the presence of a particular TLR4 allele in the nucleic acid of a population of subjects of this invention and performing a statistical analysis of the association of the TLR4 alleles with the presence of an invasive mold infection and/or impaired immune response and/or poor prognosis in the population of subjects, according to well known methods of statistical analysis. An analysis that identifies a statistical association (e.g., a significant association) between one or more particular TLR4 alleles and the presence of invasive mold infection and/or an impaired immune response and/or poor prognosis establishes a correlation between the presence of the TLR4 allele in a subject and an increased risk of developing an invasive mold infection and/or increased risk of an impaired immune response and/or poor prognosis.

For example, the identification of a TLR4 allele of this invention in a sample (e.g., a biological sample such as blood, cells, body fluid and/or tissue) can be determined using any of a variety of genotyping techniques known in the art, as described below. As used herein, the terms “genotype” or genotyping” mean the examination of a nucleic acid sample of a subject (e.g., “testing” the sample) to detect an allele or other polymorphism or mutation, as well as to identify the genetic makeup of the subject, i.e., what specific alleles are present in a nucleic acid sample from a subject. In particular, a subject of the present invention is genotyped to identify which alleles or haplotypes are present in the TLR4 gene of the subject in order to determine if the subject is at increased risk of invasive mold infection and/or increased risk of impaired immune function and/or poor prognosis due to the presence in the TLR4 gene of the subject of an allele or haplotype of this invention that has been identified to be associated with increased risk of invasive mold infection and/or increased risk of impaired immune function and/or poor prognosis.

Thus, the present invention also provides a method of identifying a SNP allele or a haplotype in the TLR4 gene that is associated with increased risk of invasive mold infection (e.g., in a transplant recipient) comprising: a) identifying the alleles of one or more single nucleotide polymorphisms in the TLR4 gene in a subject with invasive mold infection; and b) correlating the presence of the allele(s) identified in (a) with the incidence of invasive mold infection in the subject, thereby identifying a SNP allele or a haplotype in the TLR4 gene that is associated with increased risk of invasive mold infection.

Also provided herein is a method of identifying a single nucleotide polymorphism in the TLR4 gene correlated with increased risk of invasive mold infection, comprising: a) identifying a subject having an invasive mold infection; b) detecting in the subject the presence of an allele of a single nucleotide polymorphism in the TLR4 gene; and c) correlating the presence of the allele of the single nucleotide polymorphism of step (b) with the invasive mold infection in the subject, thereby identifying an allele in the single nucleotide polymorphism in the TLR4 gene correlated with increased risk of invasive mold infection.

In additional embodiments, the present invention provides a method of correlating an allele of a single nucleotide polymorphism in the TLR4 gene of a subject with increased risk of invasive mold infection, comprising: a) identifying a subject having an invasive mold infection; b) determining the nucleotide sequence of the TLR4 gene of the subject of (a); c) comparing the nucleotide sequence of step (b) with the nucleotide sequence of the TLR4 gene of a subject without an invasive mold infection; d) identifying an allele of a single nucleotide polymorphism in the nucleotide sequence of (b) that occurs more frequently than in the nucleotide sequence of a subject without an invasive mold infection; and e) correlating the allele of the single nucleotide polymorphism of (d) with the presence of invasive mold infection in the subject of (a), thereby correlating an allele of a single nucleotide polymorphism in the TLR4 gene of a subject with increased risk of invasive mold infection.

Further provided herein is a method of screening for a single nucleotide polymorphism or haplotype in the TLR4 gene of a human subject that is associated with an increased risk of invasive mold infection, mortality and/or impaired immune response, comprising detecting single nucleotide polymorphism(s) or haplotype(s) in the TLR4 gene of a human subject, performing a population based study to detect the single nucleotide polymorphism(s) or haplotype(s) in a group of human subjects with invasive mold infection, increased mortality and/or impaired or altered immune function and in ethnically matched controls, and identifying an allele or set of alleles of one or more single nucleotide polymorphisms or haplotypes in the TLR4 gene that are associated with increased risk of invasive mold infection, mortality and/or impaired immune response

For the methods of this invention, the genotyping of nucleic acid, as well as the detection of an allele in the TLR4 gene of this invention (GenBank® Accession Nos. NM_{—}138554, NT_{—}008470 and NP_{—}612564) can be carried out according to various protocols standard in the art for identifying specific nucleotides in a nucleotide sequence, as well as identifying an allele of a single nucleotide polymorphism in a gene, and as described in the Examples section provided herein. Single nucleotide polymorphisms of the TLR4 gene that are known in the art and/or are identified can be analyzed according to the methods of this invention and employing art-known protocols for identifying alleles associated with the increased risks described herein.

For example, nucleic acid can be obtained from any suitable sample from the subject that will contain nucleic acid and the nucleic acid can then be prepared and analyzed according to well-established protocols for the presence of genetic markers according to the methods of this invention. In some embodiments, analysis of the nucleic acid can be carried by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides (e.g., that are complementary to the nucleotide sequence of the TLR4 gene and/or coding sequence) for use as primers and/or probes for detecting and/or identifying genetic markers according to the methods of this invention.

The genetic markers of this invention are correlated with invasive mold infection and/or mortality as described herein according to methods well known in the art and as disclosed in the Examples provided herein for correlating genetic markers with various phenotypic traits, including disease states and pathological conditions and levels of risk associated with developing a disease or pathological condition. In general, identifying such correlation involves conducting analyses that establish a statistically significant association and/or a statistically significant correlation between the presence of a genetic marker or a combination of markers and the phenotypic trait in a population. An analysis that identifies a statistical association (e.g., a significant association) between the marker or combination of markers and the phenotype in the population establishes a correlation between the presence of the marker or combination of markers in a subject and the particular phenotype being analyzed. Such studies can be carried out in a number of subjects, who can be analyzed as a general population as well as according to gender, ethnicity, heterozygosity of an allele, homozygosity of an allele or any combination thereof, according to art-known methods.

The present invention further provides kits suitable for use in identifying a TLR4 allele and/or haplotype of this invention in a nucleic acid sample. Such kits can include, for example, reagents (e.g., probes or primers) necessary to carry out genotyping, as are well known in the art.

In carrying out the methods of this invention, detection reagents can be developed and used to identify any allele of the present invention individually or in combination with the identification of other alleles, and such detection reagents can be readily incorporated into one of the established kit or system formats that are well known in the art. The terms “kits” and “systems,” as used herein refer, e.g., to combinations of multiple allele detection reagents, or one or more allele detection reagent in combination with one or more other types of elements or components (e.g., other types of biochemical reagents, buffers, containers, packages such as packaging intended for commercial sale, substrates to which allele detection reagents are attached, electronic hardware components, etc.) Accordingly, the present invention further provides allele detection/identification kits and systems, including but not limited to, packaged probe and primer sets (e.g., TAQMAN probe/primer sets), arrays/microarrays of nucleic acid molecules, and/or beads that contain one or more probes, primers, or other detection reagents for detecting/identifying one or more alleles of the present invention. The kits/systems can optionally include various electronic hardware components; for example, arrays (“DNA chips”) and microfluidic systems (“lab-on-a-chip” systems) provided by various manufacturers. Other kits/systems (e.g., probe/primer sets) may not include electronic hardware components, but can be comprised of, for example, one or more detection reagents (along with, optionally, other biochemical reagents) packaged in one or more containers.

In some embodiments, a kit of this invention typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, etc.) necessary to carry out an assay or reaction, such as amplification and/or detection of an allele-containing nucleic acid molecule. In some embodiments of the present invention, kits are provided that contain the necessary reagents to carry out one or more assays to detect one or more alleles disclosed herein. In some embodiments of the present invention, allele detection kits/systems are in the form of nucleic acid arrays, or compartmentalized kits, including, e.g., microfluidic/lab-on-a-chip systems.

Allele detection kits/systems of this invention can contain, for example, one or more probes, or pairs of probes, that hybridize to a nucleic acid molecule at or near each target allele position. Multiple pairs of allele-specific probes can be included in the kit/system to simultaneously assay large numbers of alleles, at least one of which is an allele of the present invention. In some kits/systems, the allele-specific probes can be immobilized to a substrate such as an array or bead. The terms “arrays,” “microarrays,” and “DNA chips” are used herein interchangeably to refer to an array of distinct polynucleotides affixed to a substrate, such as glass, plastic, paper, nylon and/or other type of membrane, filter, chip, and/or any other suitable solid support. The polynucleotides can be synthesized directly on the substrate, or synthesized separate from the substrate and then affixed to the substrate. In one embodiment, the microarray can be prepared and used according to the methods described, e.g., in U.S. Pat. No. 5,837,832, U.S. Pat. No. 5,807,522, PCT Publication No. WO 95/11995, Lockhart et al. (1996) Nat. Biotech. 14:1675-1680; and Schena et al. (1996) Proc. Natl. Acad. Sci. 93:10614-10619, all of which are incorporated herein in their entireties by reference.

Any number of probes, such as allele-specific probes, can be implemented in an array, and each probe or pair of probes can hybridize to a different allele position. Polynucleotide probes can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs fixed to a solid support. Exemplary polynucleotides can be about 6-100 nucleotides in length in some embodiments, about 15-30 nucleotides in length in other embodiments, and about 18-25 nucleotides in length in yet other embodiments of this invention. For certain types of microarrays or other detection kits/systems, oligonucleotides that are only about 7-20 nucleotides in length can be used. In other types of arrays, such as arrays used in conjunction with chemiluminescence detection technology, probe lengths can be, for example, about 15-80 nucleotides, about 50-70 nucleotides in length, about 55-65 nucleotides in length, and/or about 60 nucleotides in length. The microarray or detection kit can contain polynucleotides that cover the known 5′ or 3′ sequence of a gene/transcript or target allele site, sequential polynucleotides that cover the full-length sequence of a gene/transcript; and/or unique polynucleotides selected from particular areas along the length of a target gene/transcript sequence.

Hybridization assays based on polynucleotide arrays rely on the differences in hybridization stability of the probes to perfectly matched and mismatched target sequence variants. For SNP genotyping, stringency conditions used in hybridization assays can be high enough such that nucleic acid molecules that differ from one another at as little as a single SNP position can be differentiated [e.g., typical SNP hybridization assays are designed so that hybridization will occur only if one particular nucleotide (i.e., a particular allele) is present at a SNP position, but will not occur if an alternative nucleotide (i.e., alternative allele) is present at that SNP position]. Such high stringency conditions can be used, for example, in nucleic acid arrays of allele-specific probes for SNP detection. Such, high stringency conditions are well known to those skilled in the art and can be found in, for example, Current Protocols in Molecular Biology, John Wiley & Sons, N.Y. (1989).

In other embodiments, the arrays can be used in conjunction with chemiluminescence detection technology, as is known in the art (see, e.g. U.S. Pat. Nos. 6,124,478, 6,107,024, 5,994,073, 5,981,768, 5,871,938, 5,843,681, 5,800,999, and 5,773,628, which describe methods and compositions for performing chemiluminescence detection; and USPTO Publication No. 2002/0110828, which discloses methods and compositions for microarray controls. All of these references are incorporated herein in their entireties by reference.).

A polynucleotide probe can be synthesized on the surface of the substrate by using a chemical coupling procedure and an ink jet application apparatus, as described, for example, in PCT Publication No. WO 95/251116, which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical and/or chemical bonding procedures. An array, such as described above, can be produced by hand or by using available devices (slot blot or dot blot apparatus), materials (any suitable solid support), and/or machines (including robotic instruments), and may contain: 8, 24, 96, 384, 1536, 6144 or more polynucleotides, or any other number which lends itself to the efficient use of commercially available instrumentation.

Using such arrays and/or other kits/systems, the present invention provides methods of identifying the alleles disclosed herein in a biological test sample. Such methods typically involve incubating a sample containing nucleic acid with an array comprising one or more probes corresponding to at least one allele of the present invention, and assaying for binding of a nucleic acid from the test sample with one or more of the probes. Conditions for incubating a detection reagent (or a kit/system that employs one or more such detection reagents) with a test sample vary. Incubation conditions depend on such factors as the format employed in the assay, the detection methods employed, and/or the type and nature of the detection reagents used in the assay. One skilled in the art will recognize that any one of the commonly available hybridization, amplification and array assay formats can readily be adapted to detect the alleles of this invention as disclosed herein.

A detection kit/system of the present invention can include components that are used to prepare nucleic acids from a test sample for the subsequent amplification and/or detection of an allele-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens. The test samples used in the above-described methods will vary based on such factors as the assay format, nature of the detection method, and the specific tissues, cells or extracts used as the test sample to be assayed. Methods of preparing nucleic acids, proteins, and cell extracts are well known in the art and can be readily adapted to obtain a sample that is compatible with the system utilized. Automated sample preparation systems for extracting nucleic acids from a test sample are commercially available (e.g., Qiagen's BIOROBOT 9600, Applied Biosystems' PRISM 6700, and Roche Molecular Systems COBAS AmpliPrep System).

Another form of kit included in the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other allele detection reagent for detecting one or more alleles of the present invention, one or more containers which contain wash reagents (such as phosphate buffered saline, Tris-buffers, etc.), and one or more containers which contain the reagents used to reveal the presence of the bound probe or other allele detection reagents. The kit can optionally further comprise compartments and/or reagents for, e.g., nucleic acid amplification or other enzymatic reactions such as primer extension reactions, hybridization, ligation, electrophoresis (preferably capillary electrophoresis), mass spectrometry, and/or laser-induced fluorescent detection. The kit can also include instructions for using the kit. Exemplary compartmentalized kits include microfluidic devices known in the art (e.g., Weigl et al. (2003) “Lab-on-a-chip for drug development” Adv Drug Deliv Rev. 55(3):349-77). In such microfluidic devices, the containers may be referred to as, for example, microfluidic “compartments,” “chambers,” or “channels.”

Microfluidic devices, which may also be referred to as “lab-on-a-chip” systems, biomedical micro-electro-mechanical systems (bioMEMs), or multicomponent integrated systems, are exemplary kits/systems of the present invention for analyzing alleles. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more alleles of the present invention. One example of a microfluidic system is disclosed in U.S. Pat. No. 5,589,136, which describes the integration of PCR-amplification and capillary electrophoresis in chips and which is incorporated by reference herein in its entirety. Exemplary microfluidic systems comprise a pattern of microchannels designed onto a glass, silicon, quartz, or plastic wafer included on a microchip. The movements of the samples can be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. No. 6,153,073 and U.S. Pat. No. 6,156,181.

For genotyping alleles of this invention, an exemplary microfluidic system may integrate, for example, nucleic acid amplification, primer-extension, capillary electrophoresis, and a detection method such as laser induced fluorescence detection. In a first step of such an exemplary system, nucleic acid samples are amplified, preferably by PCR. Then, the amplification products are subjected to automated primer extension reactions using ddNTPs (specific fluorescence for each ddNTP) and the appropriate oligonucleotide primers to carry out primer extension reactions that hybridize just upstream of the targeted allele. Once the extension at the 3′ end is completed, the primers are separated from the unincorporated fluorescence ddNTPs by capillary electrophoresis. The separation medium used in capillary electrophoresis can be, for example, polyacrylamide, polyethylene glycol or dextran. The incorporated ddNTPs in the single nucleotide primer extension products are identified by laser-induced fluorescence detection. Such an exemplary microchip can be used to process, for example, at least 96 to 384 samples, or more, in parallel.

As noted above, any of a variety of suitable techniques can be employed in the methods of this invention for detection of an allele of this invention. Such techniques can include, for example, the use of microsatellite array analysis, restriction fragment length polymorphism (RFLP) analysis, mass spectrometry (see also Ye et al., Hum. Mutat. 17(4):305 (2001), Chen et al., Genome Res. 10:549 (2000)), nanotechnology protocols for genomic characterization and any other protocol or technique now known or later developed for use in identifying genomic characteristics, including any of a variety of single nucleotide polymorphism (SNP) detection techniques now known or later developed.

In particular, for the identification of single-nucleotide polymorphisms (SNPs) in nucleic acid, various methods can be used, including, but not limited to, fluorescence-based sequencing, hybridization high-density variation-detection DNA chips, high performance liquid chromatography, allele-specific oligonucleotide hybridization (ASOH), nick translation PCR, PCR-ELISA ASO I0 typing, dynamic allele-specific hybridization (DASH), allele-specific inverse PCR (ASIP), inverse PCR-RFLP (IP-RFLP), single stranded conformational polymorphism (SSCP) genotyping, bi-directional PCR amplification of specific allele (bi-PASA), high-throughput SNP genotyping, homogeneous allele-specific PCR based SNP genotyping, molecular inversion probe genotyping, amplification refractory mutation system (ARMS), locked nucleic (LN) SNP genotyping, molecular beacon sequence analysis, high performance multiplex SNP analysis, amplified fragment length polymorphism (AFLP), melting curve analysis of SNPs, tetra-primer ARMS-PCR, ligase chain reaction, allele-specific polymerase chain reaction; Tₘ shift genotyping, and minisequencing.

“Single nucleotide polymorphism” or “SNP” refers to single-base pair variations within the genetic code of the individuals of a population. SNPs, which are defined in relation to a population, are variations in DNA at a single base that are found in at least 1% of the population. The terms “biallelic marker,” “marker,” “polymorphism” and “allele” are also used to denote variations at a single base and are used interchangeably. SNPs and other alleles can be identified de novo through population analysis or can be selected from numerous databases including the National Center for Biotechnology Information (NCBI) SNP database (dbSNP), the SNP Consortium (TSC) database, Human Genome Variation Database (HGVbase), and the ABI database (Applied Biosystems, Foster City, Calif.).

The term “genotype” is used herein to mean a specific allele or alleles an individual carries at a given locus. It can also be used to describe a set of alleles for multiple loci.

Also as used herein, a “haplotype” is a set of alleles on a single chromatid that are statistically associated. It is thought that these associations, and the identification of a few alleles of a haplotype block, can unambiguously identify most other polymorphic sites in its region. Such information is very valuable for investigating the genetics behind common diseases and is collected by the International HapMap Project. The term “haplotype” is also commonly used to describe the genetic constitution of individuals with respect to one member of a pair of allelic genes; sets of single alleles or closely linked genes that tend to be inherited together.

The term “phenotype” is used herein to mean the form taken by some character (or group of characters) in a specific individual. It can also mean the detectable outward manifestations of a specific genotype.

An “allele” as used herein refers to one of two or more alternative forms of a nucleotide sequence at a given position (locus) on a chromosome. Usually alleles are nucleotide sequences that make up the coding sequence of a gene, but sometimes the term is used to refer to a nucleotide sequence in a non-coding sequence. An individual's genotype for a given gene is the set of alleles it happens to possess.

The term “allele frequency” is used to mean a measure of the commonness of an allele in a population; the proportion of all alleles of that gene or polymorphism in the population that are of this specific type.

The term “Hardy-Weinberg” is used to refer to calculating the Hardy-Weinberg equilibrium for genotypes, whereby the stable frequency distribution of genotypes AA, Aa, and aa, in the proportions p², 2pq and q², respectively (where p and q are the frequencies of the alleles A and a), which is a consequence of random mating in the absence of mutation, migration, natural selection or random drift.

The term “p-value” is used herein to mean the probability that the results were not significant. For example, a p-value of 0.05 means that there are 5 chances in 100 that the results are not significant.

The term “SEM” is used to mean the standard of the mean.

The term “linkage disequilibrium” is used herein to refer to the relationship that is said to exist between an allele found at a single polymorphic site and alleles found at nearby polymorphisms if the presence of one allele is strongly predictive of the alleles present at the nearby polymorphic sites. Thus, the existence of linkage disequilibrium (LD) enables an allele of one polymorphic marker to be used as a surrogate for a specific allele of another. Furthermore, as used herein, the term “linkage disequilibrium” or “LD” refers to the occurrence in a population of two linked alleles at a frequency higher or lower than expected on the basis of the allele frequencies of the individual genes. Thus, linkage disequilibrium describes a situation where alleles occur together more often than can be accounted for by chance, which indicates that the two alleles are physically close on a DNA strand.

The term “polynucleotide” refers to a chain of nucleotides without regard to length of the chain.

Also as used herein, “linked” describes a region of a chromosome that is shared more frequently in family members or members of a population affected by a particular trait, disease or disorder, than would be expected or observed by chance, thereby indicating that the gene or genes or other identified marker(s) within the linked chromosome region contain or are associated with an allele that is correlated with the presence of a trait, disease or disorder, or with an increased or decreased risk or likelihood of the trait, disease or disorder. Once linkage is established, association studies (linkage disequilibrium) can be used to narrow the region of interest or to identify the marker (e.g., allele or haplotype) correlated with the trait, disease or disorder.

The term “genetic marker” as used herein refers to a region of a nucleotide sequence (e.g., in a chromosome) that is subject to variability (i.e., the region can be polymorphic for a variety of alleles). For example, a single nucleotide polymorphism (SNP) in a nucleotide sequence is a genetic marker that is polymorphic for two (or in some cases, three or four) alleles. SNPs can be present within a coding sequence of a gene, within noncoding regions of a gene and/or in an intergenic (e.g., intron) region of a gene. A SNP in a coding region in which both allelic forms lead to the same polypeptide sequence is termed synonymous (i.e., a silent mutation) and if a different polypeptide sequence is produced, the alleles of that SNP are non-synonymous. SNPs that are not in coding regions (i.e., in noncoding regions or regulatory regions) can still have effects on gene splicing, transcription factor binding and/or the sequence of the non-coding RNA.

Other examples of genetic markers of this invention can include but are not limited to haplotypes (i.e., combinations of alleles), microsatellites, restriction fragment length polymorphisms (RFLPs), repeats (i.e., duplications), insertions, deletions, etc., as are well known in the art.

As used herein, “nucleic acids” encompass both RNA and DNA, including cDNA, genomic DNA, mRNA, synthetic (e.g., chemically synthesized) DNA and chimeras of RNA and DNA. The nucleic acid can be double-stranded (i.e., the sequence and its complementary sequence) or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities and/or increased resistance to nucleases.

An “isolated nucleic acid” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about five or six nucleotides to about, 25, 50, 75 or 100 nucleotides, for example, about 15 to 30 nucleotides, or about 20 to 25 nucleotides, which can be used, for example, as a primer in a PCR amplification or as a probe in a hybridization assay or in a microarray. Oligonucleotides can be natural or synthetic, e.g., DNA, RNA, modified backbones, etc. Peptide nucleic acids (PNAs) can also be used as probes in the methods of this invention.

The present invention further provides a method of identifying an effective treatment regimen for a subject with an invasive mold infection, comprising correlating the presence of one or more alleles of the TLR4 gene of this invention with an effective treatment regimen for an invasive mold infection.

Thus, the methods of this invention can be used to identify subjects most suited to therapy with particular pharmaceutical agents, e.g., to prophylactically treat a subject at increased risk of developing an invasive mold infection and/or at increased risk of an impaired immune response and/or a poor prognosis. Thus, the present invention further provides a method of identifying a patient in need of such prophylactic treatment, comprising detecting a TLR4 allele of this invention in the subject. Similarly, the identification of a TLR4 allele of this invention in a subject can be used to exclude patients from certain surgeries, procedures and/or treatments due to he patient's increased likelihood of developing an invasive mold infection and/or increased likelihood of an impaired immune response and/or poor prognosis.

Thus, in further embodiments, the present invention provides a method of identifying a subject who is not suitable for surgery, an invasive procedure, a transplant and/or a treatment that increases the likelihood of the development of an invasive mold infection and/or increases the likelihood of mortality and or increases the likelihood of an impaired or altered immune response in the subject, comprising detecting a TLR4 allele of this invention in the subject. The methods of this invention can also be employed in other pharmacogenomics analyses to assist the drug development and selection process. (Linder et al. (1997) Clinical Chemistry 43:254; Marshall (1997) Nature Biotechnology 15:1249; International Patent Publication No. WO 97/40462; Schafer et al. (1998) Nature Biotechnology 16:3).

In particular, preoperative and/or pretransplant screening for the TLR4 alleles of this invention in a subject enables clinicians to better stratify a given patient for therapeutic intervention, either with drug therapy or with other modalities. Additionally, knowledge of TLR4 genotype allows patients to choose, in a more informed way in consultation with their physician, medical versus procedural therapy. Identifying the TLR4 genotype of patients who decide to or must undergo surgery or other invasive procedures enables health care providers to design altered therapeutic strategies aimed at preventing the incidence of invasive mold infection and/or mortality and/or impaired or altered immune function in patients with the TLR4 allele(s) of this invention that impart increased risk. In addition, identifying the TLR4 genotype in patients who have already experienced invasive mold infection and/or impaired or altered immune function, or who have a relative develop invasive mold infection and/or experience impaired or altered immune function, can also lead to alteration or modification in the therapeutic strategy so as to be more aggressive and proactive.

As indicated above, preoperative and/or preprocedural genotype testing can refine risk stratification and improve patient outcome. Based on the genetic risk factors identified, drugs already available and used to minimize the risk of invasive mold infection can be useful in reducing infection risk in acute settings, for example, in transplantation recipients. TLR4 genotyping can facilitate individually tailored medical therapy (personalized medicine) designed to reduce infection risk and associated morbidity and mortality and/or impaired or altered immune function. Perioperative screening can facilitate alterations in the usual course of the surgical procedure with institution of procedures designed to additionally reduce this risk.

Thus, the present invention further provides a method of identifying an effective treatment regimen for a subject with an invasive mold infection, comprising: a) correlating the presence of one or more TLR4 alleles of this invention in a population of test subjects with an invasive mold infection for whom an effective treatment regimen has been identified; and b) detecting the one or more alleles of step (a) in the subject, thereby identifying an effective treatment regimen for the subject.

Further provided is a method of correlating a TLR4 allele of this invention with an effective treatment regimen for invasive mold infection, comprising: a) detecting in a population of test subjects with an invasive mold infection and for whom an effective treatment regimen has been identified, the presence of one or more TLR4 alleles of this invention; and b) correlating the presence of the one or more alleles of step (a) with an effective treatment regimen for invasive mold infection.

Examples of treatment regimens for invasive mold infection, such as antifungal therapy, are well known in the art.

Patients who respond well to particular treatment protocols can be analyzed for specific TLR4 alleles and a correlation can be established according to the methods provided herein. Alternatively, patients who respond poorly to a particular treatment regimen can also be analyzed for particular TLR4 alleles correlated with the poor response. Then, a subject who is a candidate for treatment for an invasive mold infection can be assessed for the presence of the appropriate TLR4 allele and the most appropriate treatment regimen can be provided.

In some embodiments, the methods of correlating TLR4 alleles with treatment regimens can be carried out using a computer database. Thus the present invention provides a computer-assisted method of identifying a proposed treatment for invasive mold infection. The method involves the steps of (a) storing a database of biological data for a plurality of patients, the biological data that is being stored including for each of said plurality of patients (i) a treatment type, (ii) at least one TLR4 allele associated with invasive mold infection and (iii) at least one disease progression measure for invasive mold infection from which treatment efficacy can be determined; and then (b) querying the database to determine the dependence on said TLR4 allele of the effectiveness of a treatment type in treating invasive mold infection, to thereby identify a proposed treatment as an effective treatment for a subject carrying an TLR4 allele correlated with invasive mold infection.

In one embodiment, treatment information for a patient is entered into the database (through any suitable means such as a window or text interface), TLR4 allele information for that patient is entered into the database, and disease and/or infection information is entered into the database. These steps are then repeated until the desired number of patients has been entered into the database. The database can then queried to determine whether a particular treatment is effective for patients carrying a particular allele, not effective for patients carrying a particular allele, etc. Such querying can be carried out prospectively or retrospectively on the database by any suitable means, but is generally done by statistical analysis in accordance with known techniques, as described herein.

The present invention is more particularly described in the following examples that are intended as illustrative only since numerous modifications and variations therein will be apparent to those skilled in the art.

### EXAMPLES

### Example I

### Patients

The initial study was a cohort study of 336 patients with available DNA who underwent their first allogenic HCT from an unrelated donor after myeloablative conditioning regimen at the Fred Hutchinson Cancer Center (FHCRC) between 1995 and 2003. All patients received systemic antibiotic prophylaxis during the period of neutropenia, as well as prophylactic acyclovir, trimethoprim-sulfamethoxazole or dapsone, and fluconazole or itraconazole until day 75 after HCT.¹² Patients and donors gave written informed consent for data collection, cells storage and cryopreservation for batched genotyping, according to protocols approved by the Institutional Review Board of the FHCRC. The study was also approved the Western Institutional Review Board (Olympia, Wash., USA).

### DEFINITIONS

Cases of IA were identified from microbiological and histopathology reports and classified as probable or proven IA by reviewing clinical charts, according to standard definitions.¹³ Patients with possible IA or infection with mould other than Aspergillus species were excluded from the analysis.

### Single Nucleotide Polymorphisms

Minimal haplotype tagging SNPs were selected from the Innate Immunity Program in Genomic Applications database (IIPGA) for each candidate gene. Five additional SNPs that have been previously associated with human diseases were also genotyped. A total of 20 SNPs in TLR2,3,4, and 9 were analyzed with a mass spectrometry genotyping platform (Sequenom).¹⁴

### Statistical Analysis

Hardy-Weinberg equilibrium and pairwise linkage disequilibrium (LD) were calculated with the genhw and the pwld programs developed in Stata 9 (StataCorp, College Station, Tex.). An R² value >0.90 was considered to represent strong LD. Haplotypes were inferred using the expectation-maximization algorithm implemented in the DECIPHER program (S.A.G.E.).¹⁵ Rare haplotypes (frequencies <2%) were grouped. All analyses were performed in self-described Caucasian donors and/or recipients.

The probability of IA by TLR variants at 6 and 36 months after transplantation was estimated using cumulative incidence curves with censoring at the date of the last follow-up and treating death, second transplant and morphological relapse as competing risks.¹⁶ The underlying hazard of IA and death between TLR variant was compared using univariate Cox regression models, unless otherwise specified. Multivariate Cox models were used to evaluate the relative hazards associated with TLR variants, accounting for covariates associated with the endpoint in the univariate analysis (P<=0.15) or in previous studies.¹ Covariates were entered one by one in a pairwise model together with the significant TLR variant and kept in the final model if they remained significant (P<0.05) or modified the effect of the TLR variant (>10%).

Among the 20 SNPs tested, two pairs of SNPs were found to be in strong LD (TLR4 1063 A/G and TLR4 1363 C/T [r²=0.96]; TLR9+1174 G/A and TLR9 1635 A/G [r²=0.96]), as in previous observations.^{17,18} Since the analyses for these SNPs are redundant, 18 independent tests were considered for multiple testing correction, using the Bonferroni method. For haplotypes and diplotypes, 4 independent tests were considered (i.e., one for each gene).

### Cohort Study

The cohort study included 336 patients who underwent an allogenic HCT following a myeloablative conditioning regimen (Table 1). The median follow up time among patients who survived was 84 months (range 5-124). Among the 336 patients, 33 had proven or probable IA.

### Univariate Analysis

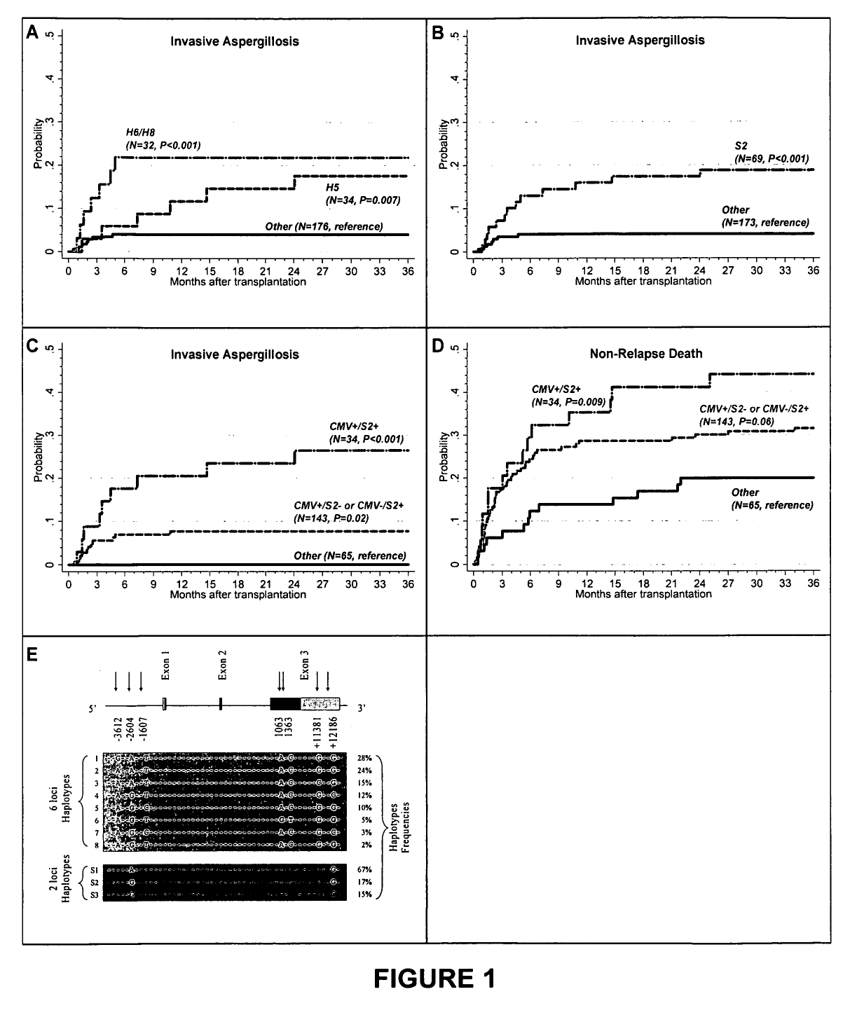

Cumulative incidence estimates were performed to assess the probability of developing IA by TLR SNPs and haplotypes, in HCT recipients and donors. Two SNPs in HCT recipients (TLR2 597 T/C [N199N] and TLR3 1234 C/T [L412F]) and 3 TLR4 SNPs in donors (−2604 A/G, 1063 A/G [D299G] and 1363 C/T [T399I]) influenced the risk of developing IA (Table 2). The cumulative incidence of IA at 30 months was 15% versus 4% for presence versus absence of TLR2 597C (P=0.06, Cox Regression), 7% versus 16% for TLR3 1234T (protective effect, P=0.03), 13% versus 4% for TLR4 −2604G (P=0.01) and 22% versus 7% for TLR4 1063G (P=0.02). No association was found for recipient or donor TLR9 SNPs. Three TLR4 haplotypes in donors (H5, H6 and H8) were associated with an increased hazard of IA (Table 5). For H5, the association was stronger when the comparison was performed over 30 months (cumulative incidence 17% versus 7% for the presence versus the absence of H5; P=0.04). For H6 and H8, the association was stronger during the first 6 months following HCT (cumulative incidence 22% versus 5% for H6, P=0.002; 22% versus 6% for H8, P=0.03). Donor H6/H8 and H5 were then entered in the same Cox regression model to assess the risk of IA (FIG. 1A). Haplotypes H6/H8 were associated with high risk (P<0.001), while H5 was associated with intermediate risk (P=0.007), compared to the reference group. These 3 haplotypes shared the presence of a G at position −2604 and a G at position +12186 (FIGS. 1B and E). Presence of this 2-loci haplotype (S2) in donor was strongly associated with the risk of IA at 30 months (19% versus 4% for the presence versus the absence of S2, P<0.001).

### Multivariate Analysis

The associations of TLR SNPs with IA remained significant in multivariate Cox regression models, when adjusted for acute GVHD and CMV serostatus (Table 3). However, when correction for multiple testing was applied, the association remained significant only for donor TLR4 1063G (HR=7.65 [2.41-24.29], P<0.001, adj. P=0.01). When the multivariate analysis was performed using haplotypes, donor TLR4H5 and H6 were still associated with IA, even after correction for multiple testing, and there was a trend toward an association between donor H8 and IA (Table 6). The HR was 2.14 (0.69-6.66, P=0.19) for a single H5 in donor and 5.19 (1.48-18.28, P=0.01, adj. P=0.04) for a pair of H5, suggesting a possible dominant effect. The HR for the presence versus the absence of H6 was 7.65 (2.41-24.29, P<0.001, adj. P=0.002). When using the 2-loci haplotypes, the presence of donor S2 was significantly associated with the risk of IA (HR=6.03 [2.38-15.30], P<0.001, adj. P<0.001). The other significant covariates in the model were a positive CMV serostatus in the patient and/or the donor (4.62 [1.51-14.11], P=0.007) and acute GVHD (HR 2.43 [1.00-5.86], P=0.05), indicating that S2 in donor, CMV seropositivity and acute GVHD were independent risk factors for IA.

Risk Assessment before HCT

Since CMV serology and TLR4 haplotypes can both be used to evaluate the risk of IA and death before HCT, the patients were stratified into 4 groups according to CMV serology (CMV− versus CMV+) and presence versus absence of S2 in donors (S2− versus S2+). S2 is generally inferable from SNPs 2604 and 12186 genotypes. Even for the double heterozygotes where diplotypes cannot be directly inferred, the probability of mis-classifying S2 was 0.7% (Table 7). The cumulative incidence of IA increased from 0% for CMV−/S2− to 26% for CMV+/S2+(P<0.001, FIG. 1C). Thus, patients could be separated into groups of high (CMV+/S2+) or intermediate risk of IA (CMV+/S2− or CMV−/S2+), compared to the reference group (CMV−/S2−). Risk comparison could not be performed using a multivariate Cox regression model, since no patient in the reference group had IA. The interaction between CMV+ and S2+ did not influence the risk of IA (P=0.95). The cumulative incidence of non-relapse death increased from 20% for CMV−/S2- to 44% for CMV+/S2+ (P=0.009, log-rank test, FIG. 1D). The hazard ratio was 2.47 ([1.10-5.56], P=0.03) for CMV+/S2+, and 1.15 ([0.57-2.33], P=0.70) for CMV+/S2− and CMV−/S2+, compared to CMV−/S2− patients, after adjusting for pre and post-transplant covariates (Table 4).

This study shows that 2 TLR4 SNPs in HCT donors (1063G and 1363T, both in strong LD with haplotype H6) were significantly associated with IA in HCT recipients. These SNPs were part of a 2-loci TLR4 haplotype (S2, haplotype frequency 17%), whose presence in HCT donors was also associated with an increased risk of developing IA. The results are strengthened by the fact that the involvement of TLR4 polymorphisms in IA is biologically highly plausible.

TLRs were initially identified through their homology with the Toll gene in Drosophila melanogaster, which triggers the production of drosomycin and is required for effective protection against fungal infections¹⁹. Mammalian TLR4 was first recognized as the transmembrane receptor for lipopolysaccharide (LPS), a key component for the detection of Gram-negative bacteria.²⁰ Subsequent studies have shown that TLR4 is also involved in the recognition of fungal ligands, including mannan (Candida albicans) ²¹ and glucuronoxylomannan (Cryptococcus neoformans).²² Several studies have highlighted the role of TLR2 and TLR4 in the innate immune recognition of Aspergillus species, though the microbial ligand has not yet been identified.⁵⁻¹⁰

TLR4 1063G and 1363T SNPs have been previously associated with susceptibility to infections caused by Gram-negative bacteria,^{23,24} Candida albicans,²⁵ Brucella species,²⁶ respiratory syncytial virus²⁷ and Plasmodium falciparum.²⁸ Individuals heterozygous for the 1063G and 1363T alleles were hyporesponsive to LPS as measured by bronchospastic response after inhalation of endotoxin.²⁹ Furthermore, airway epithelial cells isolated from heterozygous individuals had deficient response to LPS, suggesting that 1063G and 1363T act in a dominant fashion with respect to the wild-type allele²⁹. However, monocytes and whole blood isolated from heterozygous subjects did not show abnormal responses to LPS^{30,31}, suggesting that the effects of these mutations may vary between cell types or experimental conditions, or that polymorphisms other than 1063G and 1363T interfere with the results.

While several studies evaluated the role of TLR4 1063G and 1363T with regard to human disease, few studies have addressed the role of TLR4 haplotypes. The present study demonstrates that TLR4 1063G and 1363T are contained in a 2-loci haplotype (S2), grouping the H5, H6 and H8 haplotypes. All episodes of late IA (>6 months after transplantation, almost exclusively found in the cohort study) occurred in donor H5 carriers; despite the rare frequency of donor H8 (2%), both studies showed a trend toward its association with the risk of IA. Although H6 had a stronger effect on IA than H5 and H8, these observations taken together suggest that S2 is not just a marker for H6, but that SNPs in H5 and H8 may also affect stability or processing of the gene transcripts, and influence gene expression and/or splicing.³²

The identification of donors associated with high risk of severe infections has direct implications for the prevention strategies in allogenic HCT patients. Since CMV serology and TLR4 haplotypes can both be assessed before HCT, patients could be stratified into high risk (CMV+/S2+), intermediate risk (CMV+/S2− or CMV−/S2+) and low risk groups (CMV−/S2−) of IA and death (FIGS. 1C and D). The former can benefit from specific surveillance and/or empiric prophylaxis or pre-emptive therapy, while the latter may not require such treatments. Alternatively, since the polymorphisms at risk are in the donor in the present study, future HCT donors can be screened for genetic polymorphisms associated with the risk of infections. When several donors are available, those with polymorphisms predicting a low risk of infection (S2−) might be preferentially selected.

The present study identified a strong association between TLR4 haplotypes in unrelated donors and the risk of IA in a cohort of allogenic HCT recipients. These results demonstrate the feasibility of a novel method to risk-stratify allogenic HCT recipients for important infectious complications.

### Example II

### Patients

All patients who received an allogenic transplant from the Fred Hutchinson Cancer Research Center/Seattle Cancer Care Alliance between 1989 and 2004 and consented to genetic studies were eligible for this analysis. Recovery of specimens and extraction of DNA by our Human Immunogenetics Program (HIP) is an ongoing process and solely based upon the availability of specimens, with no preferences given to any specific clinical parameters. Selection of patients for the discovery and validation studies was done sequentially from the overall pool of specimens available at the time the respective studies were conducted, and no patient was included in both studies.

For the discovery study, 374 patients with available DNA who underwent their first allogenic HCT from an unrelated donor between 1995 and 2003 were identified. Cases were identified from microbiological and histopathology reports in clinical charts and classified as “probable” or “proven” IA, using definitions developed by the Mycoses Study Group and European Organization for Research and Treatment of Cancer.¹³ Among the 374 patients, 38 were excluded for at least one of the following reasons: both donor and recipient had unknown or non-Caucasian ethnicity (N=32), IA occurred before the 1^{st} or after a 2^{nd} transplantation (N=5), the diagnosis of IA was classified as “possible” (N=3), or infection was due to an invasive mold other than Aspergillus species (N=2).

For the validation study, a matched case control design was used, which included HCT from related donors. A minimal number of 100 cases with at least one matched control was required to detect odd ratios of ˜2.3, ˜2.0 and ˜1.8 with 80% power and 5% type I error rate, for allelic frequencies of 10%, 20% and 30%, respectively (calculated with Quanto 1.2.3).³³ From 223 cases of IA that occurred between 1989 and 2004 from the overall HIP pool, 120 were excluded for one of the following reasons: donor had unknown or non-Caucasian ethnicity (N=67), IA occurred before the 1^{st} or after a 2^{nd} transplant (N=37), the diagnosis of IA was classified as “possible” (N=16). For each of the 103 cases of IA, 1 to 6 controls were matched for patient age (<40 or >=40), donor ethnicity, donor type and year (+/−5 years) of transplantation. Out of 404 selected controls, 263 had available DNA and were utilized for the validation study. A subgroup analysis was performed to account for differences in HCT types.

Graft-versus-host disease (GVHD) and CMV disease were diagnosed according to standard definitions³⁴⁻³⁶. All patients received systemic antibiotic prophylaxis during the period of neutropenia, as well as prophylactic acyclovir, trimethoprim-sulfamethoxazole or dapsone, and fluconazole until day 75 after HCT. A small number of patients (35 in the discovery and 13 in the validation study) received itraconazole or voriconazole prophylaxis based on individual physician decision, or as a part of a prospective randomized study of the efficacy of itraconazole versus fluconazole prophylaxis.¹² Patients and donors gave written informed consent for data collection, cell storage and cryopreservation for batched genotyping, according to protocols approved by the Institutional Review Board of the FHCRC. The study was also approved by the Western Institutional Review Board (Olympia, Wash., USA).

### Single Nucleotide Polymorphisms

Previously identified minimal haplotype tagging SNPs from the Innate Immunity Program in Genomic Applications database were used for each candidate gene. Five additional SNPs that have been previously associated with human diseases were also genotyped. A total of 20 SNPs in TLR2,3,4, and 9 were identified with a mass spectrometry genotyping platform (Sequenom).¹⁴

### Statistical Analysis

Hardy-Weinberg equilibrium and pairwise linkage disequilibrium (LD) were calculated with the genhw and the pwld programs developed in Stata 9.2 (StataCorp, College Station, Tex.). An R² value >0.90 was considered to represent strong LD. Haplotypes were inferred using the expectation-maximization algorithm implemented in the DECIPHER program (S.A.G.E.).¹⁵ All rare haplotypes (frequencies <2%) were grouped into one group for each gene. All analyses were performed in self-described Caucasian donors and/or recipients.

The probability of IA by TLR variants at 6 and 36 months after transplantation was estimated using cumulative incidence curves with censoring at the date of the last follow-up and treating death, second transplant and morphological relapse as competing risks.¹⁶ The duration of 36 months was chosen because all cases of IA in the discovery cohort had occurred during this period of time and the duration of six months was chosen to assess early IA. The underlying hazard of IA and death between TLR variants was compared using univariate Cox regression models, unless otherwise specified. Multivariate Cox models were used to evaluate the relative hazards associated with TLR variants. Candidate covariates were selected from those factors associated with the endpoint in the univariate analysis (P<=0.15) or in previous studies.¹ They were entered one by one in a pairwise model together with the significant TLR variant and kept in the final model if they remained significant (P<0.05) or altered the association with the TLR variant (>10%). Conditional logistic regression was used for analysis of the case-control validation study, with similar variable selection methods employed.

Due to the large number of statistical comparisons made in the discovery study, the p-values presented should be viewed in the appropriate context. To facilitate this interpretation, tables indicate which p-values would remain significant when traditional Bonferroni corrections are applied to adjust for multiple comparisons. Among the 20 SNPs tested, two pairs of SNPs were found to be in strong LD (TLR4 1063 A/G and TLR4 1363 C/T [r²=0.96]; TLR9+1174 G/A and TLR9 1635 A/G [r²=0.96]), as in previous observations.¹⁷⁻¹⁸ As the analyses for these SNPs are redundant, 18 independent tests were considered for multiple testing correction. For haplotypes, four independent tests (i.e., one for each gene) were considered. As the case control study was performed for validation and was limited to TLR4, no correction for multiple testing was applied in this study.

### Discovery Study

The discovery study included 336 patients who underwent an allogenic HCT following a myeloablative conditioning regimen (Table 8). The median follow up time among patients who survived was 84 months (range 5-124). Among the 336 patients, 33 had proven or probable IA.

### Univariate Analysis

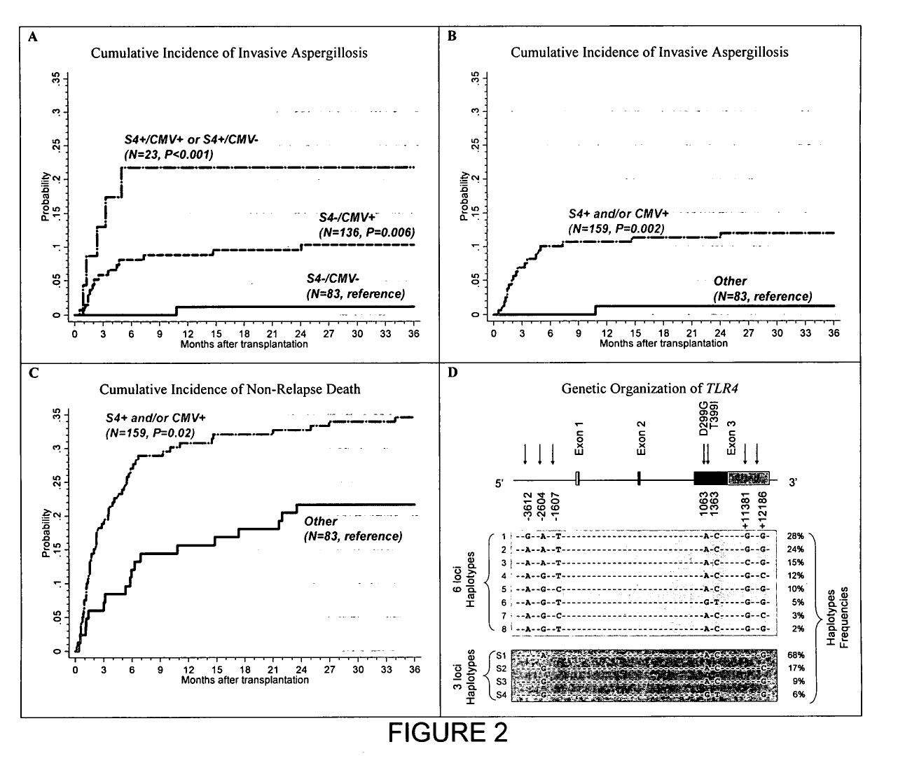

Cumulative incidence estimates at 6 and 36 months after HCT were performed to assess the probability of developing IA by TLR SNPs and haplotypes, in HCT recipients and donors. Two SNPs in HCT donors (1063 NG [D299G] and 1363 UT [T399I], both in strong LD, R²=0.96) influenced the risk of developing IA. The association was stronger when the comparison was made over 6 months (for TLR4 1363T, cumulative incidence of IA 22% versus 5%, P=0.002, Table 9) and remained present after correction for multiple testing (adj. P=0.03). Another donor SNP in TLR4 (−2604G) tended to influence the risk of IA when the comparison was made over 36 months (cumulative incidence of IA 7% versus 22%, P=0.01), but the association was no longer present when correction for multiple testing was applied (adj. P=0.20). Three TLR4 haplotypes in donors (H5, H6 and H8) were (or tended to be) associated with an increased hazard of IA. Since these three haplotypes included −2604G and +12186G carriers, and H6 also included 1363T carriers, a 3 loci haplotype was created (FIG. 2D). Haplotypes S3 and S4 influenced the risk of developing IA. For S3, the association was stronger when the comparison was made over 36 months (17% versus 6%, P=0.01, adj. P=0.04); for S4, a haplotype that included 1363T carriers, it was stronger when the comparison was made over 6 months (22% versus 5%, P=0.002, adj. P=0.03).

### Multivariate Analysis

The final SNP model included TLR4 −2604G (HR=3.22 [1.02-10.16], P=0.05, Table 10) and 1363T (HR=4.96 [1.02-10.16], P=0.008). The final haplotype model included S3 (HR=2.20 [1.14-4.25], P=0.02) and S4 (HR=6.16 [1.97-19.26], P=0.002). Both final models also included positive CMV serostatus in the patient and/or the donor and acute GVHD (HR=4.90 [1.51-15.91], P=0.008 and HR=3.11 [1.22-7.90], P=0.02, respectively, in the haplotype model), indicating that donor S4 (or TLR4 1363T), CMV seropositivity and acute GVHD were independent risk factors for IA. The interaction between CMV+ and S4+ did not influence the risk of IA (P=0.23).

### Validation Study

The characteristics of the patients in the case-control study were similar to those of the patients from the initial study (Table 8), but the validation study included recipients of both related and unrelated donors, and the median time to IA after transplantation was shorter (68 versus 100 days). The association of donor TLR4 S4 with the risk of IA was confirmed in the validation study (OR=2.63 [1.19-5.84], P=0.02). In the subgroup analysis, the association between S4 and IA was present in recipients of unrelated donors (OR=5.00 [1.04-24.01], P=0.04, Table 10), but non-significant in recipients of related donors (OR=2.29 [0.93-5.68], P=0.07). No association between S3 and IA was observed in the validation study. However, in this group, haplotype S2 tended to increase the risk of IA (OR=1.86 [1.01-3.44], P=0.05); this haplotype contained TLR4 +12186G carriers, and this allele also increased the risk of IA in the SNP model (OR=2.18 [1.13-4.21], P=0.02).

Risk Assessment before HCT

Because CMV serology and TLR4 haplotypes can both be used to evaluate the risk of IA and death before HCT, patients were stratified into groups according to CMV serology (CMV− versus CMV+) and donor haplotype S4 (noted “S4−” versus “S4+”). In the discovery study, IA occurred in one out of 83 CMV−/S4− patients (1%), 14 out of 136 CMV+/S4− patients (10%) and five out of 23 CMV−/S4+ or CMV+/S4+ patients (22%, FIG. 2A). Since the number of patients in some groups was small, all CMV+ and/or S4+ patients were grouped together (FIGS. 1B-C). The cumulative incidence of IA increased from 1% in CMV−/S4− (N=83) to 12% in CMV+ and/or S4+ (N=159, HR=11.95 [1.60-89.34], P=0.02, after adjustment for pre-transplant covariates). The cumulative incidence of non-relapse death increased from 22% for CMV−/S4− to 35% in CMV+ and/or S4+ (HR=1.88 [1.10-3.20], P=0.02, Table 11, after adjustment for pre-transplant covariates). In the validation study, the OR for the risk of IA was 3.10 (1.29-7.45, P=0.01) for CMV+/S4+ and CMV−/S4+ patients and 1.54 (0.84-2.85, P=0.16) for CMV+/S4− patients, compared to CMV−/S4− patients, after adjusting for pre and post-transplant covariates.

This study identifies an association between a TLR4 haplotype in HCT donors (S4, haplotype frequency 6%) and IA in HCT recipients. This haplotype includes carriers of two SNPs in strong LD (1063G and 1363T) that influence TLR4 function. This study also identifies an association between TLR4 haplotypes in unrelated donors and the risk of IA in two separate studies of allogenic HCT recipients. The identification of donors associated with high risk of severe infections can have direct implications for the prevention strategies in allogenic HCT patients.

The foregoing is illustrative of the present invention, and is not to be construed as limiting thereof. The invention is defined by the following claims, with equivalents of the claims to be included therein. All publications, patent applications, patents, patent publications, sequences identified by GenBank and/or SNP accession numbers, and other references cited herein are incorporated by reference in their entireties for the teachings relevant to the sentence and/or paragraph in which the reference is presented.

### REFERENCE LIST

1. Marr K A, Carter R A, Crippa F, Wald A, Corey L. Epidemiology and outcome of mould infections in hematopoietic stem cell transplant recipients. Clin Infect Dis 2002; 34(7):909-17.
2. Upton A, Kirby K A, Carpenter P, Boeckh M, Marr K A. Invasive aspergillosis following hematopoietic cell transplantation: outcomes and prognostic factors associated with mortality. Clin Infect Dis 2007; 44(4):531-40.
3. Beutler B. Inferences, questions and possibilities in Toll-like receptor signalling. Nature 2004; 430(6996):257-63.
4. Akira S, Uematsu S, Takeuchi 0. Pathogen recognition and innate immunity. Cell 2006; 124(4):783-801.
5. Braedel S, Radsak M, Einsele H, et al. Aspergillus fumigatus antigens activate innate immune cells via toll-like receptors 2 and 4. British journal of haematology 2004; 125(3):392-9.
6. Mambula S S, Sau K, Henneke P, Golenbock D T, Levitz S M. Toll-like receptor (TLR) signaling in response to Aspergillus fumigatus. The Journal of biological chemistry 2002; 277(42):39320-6.
7. Meier A, Kirschning C J, Nikolaus T, Wagner H, Heesemann J, Ebel F. Toll-like receptor (TLR) 2 and TLR4 are essential for Aspergillus-induced activation of murine macrophages. Cellular microbiology 2003; 5(8):561-70.
8. Netea M G, Warris A, Van der Meer J W, et al. Aspergillus fumigatus evades immune recognition during germination through loss of toll-like receptor-4-mediated signal transduction. J Infect Dis 2003; 188(2):320-6.
9. Gersuk G M, Underhill D M, Zhu L, Marr K A. Dectin-1 and TLRs permit macrophages to distinguish between different Aspergillus fumigatus cellular states. J Immunol 2006; 176(6):3717-24.
10. Bellocchio S, Moretti S, Perruccio K, et al. TLRs govern neutrophil activity in aspergillosis. J Immunol 2004; 173(12):7406-15.
11. Bochud P Y, Bochud M, Telenti A, Calandra T. Innate immunogenetics: a tool for exploring new frontiers in host defence. Lancet Infect Dis 2007; 7((8):531-42.
12. Marr K A, Crippa F, Leisenring W, et al. Itraconazole versus fluconazole for prevention of fungal infections in patients receiving allogeneic stem cell transplants. Blood 2004; 103(4):1527-33.
13. Ascioglu S, Rex J H, de Pauw B, et al. Defining opportunistic invasive fungal infections in immunocompromised patients with cancer and hematopoietic stem cell transplants: an international consensus. Clin Infect Dis 2002; 34(1):7-14.
14. Bochud P Y, Hersberger M, P, et al. Polymorphisms in Toll-like receptor 9 influence the clinical course of HIV-1 infection. Aids 2007; 21(4):441-6.
15. S.A.G.E. Statistical Analysis for Genetic Epidemiology. Case Western Reserve University. In. 5.0 ed; 2005.
16. Kalbfleisch J P, R L. The statistical Analysis of Failure Time Data. New-York: John Wiley & Sons; 1980.
17. Kiechl S, Lorenz E, Reindl M, et al. Toll-like receptor 4 polymorphisms and atherogenesis. The New England Journal of Medicine 2002; 347(3):185-92.
18. Lazarus R, Klimecki W T, Raby B A, et al. Single-nucleotide polymorphisms in the Toll-like receptor 9 gene (TLR9): frequencies, pairwise linkage disequilibrium, and haplotypes in three U.S. ethnic groups and exploratory case-control disease association studies. Genomics 2003; 81(1):85-91.
19. Lemaitre B, Nicolas E, Michaut L, Reichhart J M, Hoffmann JA. The dorsoventral regulatory gene cassette spatzle/Toll/cactus controls the potent antifungal response in Drosophila adults. Cell 1996; 86(6):973-83.
20. Poltorak A, He X, Smirnova I, et al. Defective LPS signaling in C3H/HeJ and C57BL/10ScCr mice: mutations in TIr4 gene. Science 1998; 282(5396):2085-8.
21. Netea M G, Gow N A, Munro C A, et al. Immune sensing of Candida albicans requires cooperative recognition of mannans and glucans by lectin and Toll-like receptors. The Journal of clinical investigation 2006; 116(6):1642-50.
22. Shoham S, Huang C, Chen J M, Golenbock D T, Levitz S M. Toll-like receptor 4 mediates intracellular signaling without TNF-alpha release in response to Cryptococcus neoformans polysaccharide capsule. J Immunol 2001; 166(7):4620-6.
23. Agnese D M, Calvano J E, Hahm S J, et al. Human toll-like receptor 4 mutations but not CD14 polymorphisms are associated with an increased risk of gram-negative infections. J Infect Dis 2002; 186(10):1522-5.
24. Lorenz E, Mira J P, Frees K L, Schwartz D A. Relevance of mutations in the TLR4 receptor in patients with gram-negative septic shock. Arch Intern Med 2002; 162(9):1028-32.
25. Van der Graaf C A, Netea M G, Morre S A, et al. Toll-like receptor 4 Asp299GlyfThr399IIe polymorphisms are a risk factor for Candida bloodstream infection. EurCytokine Netw 2006; 17(1):29-34.
26. Rezazadeh M, Hajilooi M, Rafiei A, et al. TLR4 polymorphism in Iranian patients with brucellosis. J Infect 2005.
27. Tal G, Mandelberg A, Dalal I, et al. Association between common Toll-like receptor 4 mutations and severe respiratory syncytial virus disease. The Journal of infectious diseases 2004; 189(11):2057-63.
28. Mockenhaupt F P, Cramer J P, Hamann L, et al. Toll-like receptor (TLR) polymorphisms in African children: Common TLR-4 variants predispose to severe malaria. Proc Natl Acad Sci USA 2006; 103(1):177-82.
29. Arbour N C, Lorenz E, Schutte B C, et al. TLR4 mutations are associated with endotoxin hyporesponsiveness in humans. Nat Genet 2000; 25(2):187-91.
30. von Aulock S, Schroder N W, Gueinzius K, et al. Heterozygous toll-like receptor 4 polymorphism does not influence lipopolysaccharide-induced cytokine release in human whole blood. J Infect Dis 2003; 188(6):938-43.
31. Erridge C, Stewart J, Poxton I R. Monocytes heterozygous for the Asp299Gly and Thr399IIe mutations in the Toll-like receptor 4 gene show no deficit in lipopolysaccharide signalling. Journal of Experimental Medicine 2003; 197(12):1787-91.
32. Zheng Z M. Regulation of alternative RNA splicing by exon definition and exon sequences in viral and mammalian gene expression. Journal of biomedical science 2004; 11(3):278-94.
33. Gauderman W, Morrison J. QUANTO 1.1: A computer program for power and sample size calculations for genetic-epidemiology studies. In; 2006.
34. Przepiorka D, Weisdorf D, Martin P, et al. 1994 Consensus Conference on Acute GVHD Grading. Bone Marrow Transplant 1995; 15(6):825-8.
35. Sullivan KM, Agura E, Anasetti C, et al. Chronic graft-versus-host disease and other late complications of bone marrow transplantation. Semin Hematol 1991; 28(3):250-9.
36. Ljungman P, Griffiths P, Paya C. Definitions of cytomegalovirus infection and disease in transplant recipients. Clin Infect Dis 2002; 34(8):1094-7.

TABLE 1
Characteristics of Transplant Recipients

| | Cohort Study | | |
|---|---|---|---|
| | IA (%) | No IA (%) | |
| A. Patients Characteristics | N = 33 | N = 303 | P‡ |
| Donor/Recipient Ethnicity | | | |
| Caucasian/Caucasian | (55) | (67) | |
| Caucasian/Other* | (39) | (27) | |
| Other*/Caucasian | (6) | (6) | |
| Age groups | | | |
| <19 | (15) | (12) | |
| 19-40 | (42) | (42) | 0.65 |
| >=40 | (42) | (46) | 0.63 |
| Donor/Recipient sex | | | |
| Male/Male | (36) | (40) | |
| Male/Female | (27) | (23) | 0.57 |
| Female/Male | (21) | (19) | 0.63 |
| Female/Female | (15) | (17) | 0.92 |
| Underlying Disease | | | |
| CML - chronic phase | (36) | (40) | |
| Hematologic malignancy - 1st remission | (52) | (45) | 0.32 |
| Hematologic malignancy - non-1st remission | (9) | (13) | 0.73 |
| Other** | (3) | (2) | 0.61 |
| Conditioning Regimen | | | |
| Myeloablative with Total Body Irradiation | (82) | (86) | |
| (TBI) | | | |
| Myeloablative without TBI | (18) | (14) | 0.55 |
| Graft source | | | |
| Bone marrow cells§ | (94) | (94) | |
| Peripheral blood cells | (6) | (6) | 0.97 |
| Donor/Recipient CMV serostatus | | | |
| D−/R− | (24) | (46) | |
| D−/R+, D+R+ or D+R− | (76) | (54) | 0.01 |
| Anti-GVH prophylaxis | | | |
| Containing anti-T cells antibodies | (9) | (8) | 0.92 |
| Graft versus Host Disease | | | |
| Acute (grade II-IV)-Cum. Incidence - day 80 | (97) | (88) | 0.40 |
| Chronic Extensive-Cum. Incidence - day 300 | (57) | (68) | 0.04 |
| CMV disease | | | |
| Cumulative Incidence - day 150 | (12) | (4) | 0.08 |
| Itraconazole prophylaxis | (3) | (11) | 0.20 |

| B. Invasive aspergillosis | N = 33 | % |
|---|---|---|
| Diagnosis | | |
| Proven | 16 | (48) |
| Probable | 17 | (52) |
| Localization | | |
| Pulmonary | 20 | (61) |
| Disseminated | 7 | (21) |
| Sinus | 3 | (9) |
| Other§§ | 3 | (9) |
| Microbiological documentation | | |
| Aspergillus fumigatus | 22 | (67) |
| Aspergillus niger | 3 | (9) |
| Aspergillus flavus | 1 | (3) |
| Aspergillus species | 3 | (9) |
| Mixed infection with Aspergillus species† | 2 | (6) |
| Culture Negative | 2 | (6) |
| Median time from transplantation (range) | 100 | (11-732) |

*Donor ethnicity was missing in 94 cases.
**Aplastic anemia (n = 5), paroxysmal nocturnal hemoglobinuria (n = 1) and idiopathic thrombocytopenia (n = 1).
§One patient with IA was transplanted with cord blood cells.
§§Central nervous system (n = 2); mouth (n = 1)
†A. fumigatus + A. niger (n = 1); A. fumigatus + A. flavus (n = 1); Aspergillus species + Pseudallescheria boydii + Rhizomucor (n = 1)
‡P is the P value for univariate Cox regression models. Variables with P values <0.2 were considered as potential covariates for multivariate analysis (see Tables 3 & 4).

TABLE 2
Univariate Analysis of the Association of TLR Alleles in Recipients and Donors with
Cumulative Incidence of Invasive Aspergillosis.

| | | | | | | | | | | Cumulative Incidence of IA (36 months, %) | | | | | |
|---|---|---|---|---|---|---|---|---|---|---|---|---|---|---|---|
| | | | | | | | | | | Patient (N = 316) | | | Donor (N = 242) | | |
| | Gene | | | Amino | | | | | LD | Allele | Allele | | Allele | Allele | |
| Gene | Region | SNP | | acid change | rs# | MAF | HWE | | (R²) | Absent | Present | P | Absent | Present | P |
| TLR2 | Intr. 1 | −16934 | T/A | — | rs4696480* | 0.50 | 0.12 | | | (5) | (11) | 0.11 | (3) | (10) | 0.12 |
| (4q32) | Intr. 1 | −15607 | A/G | — | rs1898830* | 0.35 | 0.78 | | | (13) | (7) | 0.09 | (7) | (9) | 0.50 |
| | Ex. 3 | 597 | T/C | N199N | rs3804099* | 0.45 | 0.49 | | | (4) | (12) | 0.06 | (7) | (9) | 0.74 |
| | Ex. 3 | 1350 | T/C | S450S | rs3804100* | 0.07 | 0.11 | | | (9) | (15) | 0.26 | (8) | (10) | 0.68 |
| | Ex. 3 | 2258 | G/A | R753Q | rs5743708 | 0.03 | 1.00 | | | (10) | | 1.00 | (8) | (18) | 0.11 |
| TLR3 | 5′ | −8921 | A/T | — | rs5743303* | 0.19 | 0.32 | | | (10) | (9) | 0.84 | (10) | (4) | 0.16 |
| (4q35) | 5′ | −8441 | T/A | — | rs5743305* | 0.36 | 0.93 | | | (9) | (10) | 0.72 | (6) | (9) | 0.39 |
| | Intr. 3 | +2602 | G/C | — | rs5743314* | 0.22 | 0.90 | | | (9) | (11) | 0.68 | (8) | (9) | 0.63 |
| | Exon 4 | 1234 | C/T | L412F | rs3775291* | 0.29 | 0.61 | | | (13) | (6) | 0.03 | (10) | (7) | 0.51 |
| TLR4 | 5′ | −3612 | A/G | — | rs2770150* | 0.27 | 0.91 | | | (10) | (9) | 0.53 | (11) | (6) | 0.18 |
| (9q32-q33) | 5′ | −2604 | A/G | — | rs10759931* | 0.34 | 1.00 | | | (9) | (10) | 0.82 | (4) | (13) | 0.02 |
| | 5′ | −1607 | T/C | — | rs10759932* | 0.13 | 0.58 | | | (10) | (8) | 0.52 | (7) | (11) | 0.31 |
| | Ex. 4 | 1063 | A/G | D299G | rs4986790 | 0.06 | 0.28 | | | (10) | (9) | 0.85 | (7) | (22) | 0.02 |
| | | | | | | | | {close oversize brace} | 0.96 | | | | | | |
| | Ex. 4 | 1363 | C/T | T399I | rs4986791 | 0.07 | 0.07 | | | (10) | (9) | 0.82 | (7) | (22) | 0.02 |
| | 3′ | +11381 | G/C | — | rs11536889* | 0.15 | 0.20 | | | (10) | (11) | 0.47 | (10) | (4) | 0.16 |
| | 3′ | +12186 | G/C | — | rs7873784* | 0.16 | 0.44 | | | (9) | (10) | 0.85 | (10) | (5) | 0.18 |
| TLR9 | 5′ | −1486 | T/C | — | rs187084* | 0.41 | 0.38 | | | (10) | (9) | 0.98 | (11) | (7) | 0.31 |
| (3p21.3) | 5′ | −1237 | T/C | — | rs5743836 | 0.16 | 0.76 | | | (11) | (7) | 0.31 | (7) | (11) | 0.21 |
| | Intr. 1 | +1174 | G/A | — | rs352139* | 0.44 | 0.49 | | | (8) | (11) | 0.62 | (6) | (9) | 0.40 |
| | | | | | | | | {close oversize brace} | 0.96 | | | | | | |
| | Ex. 2 | 1635 | A/G | P545P | rs352140 | 0.45 | 0.49 | | | (8) | (11) | 0.73 | (7) | (9) | 0.45 |

IA stands for invasive aspergillosis,
SNP for single nucleotide polymorphism,
MAF for minor allele frequency,
HWE for Hardy-Weinberg equilibrium test,
LD for linkage disequilibrium.
P is the P value for univariate Cox regression model.
Minor allele frequencies, Hardy-Weinberg equilibrium tests and linkage disequilibrium are calculated together for both Caucasian patients and donors.
*indicate haplotype tagging SNPs. Since 20 out of 336 patients and 94 out of 336 donors were not Caucasian or had missing ethnicity, the denominators in the cohort study were 316 patients and 242 donors.
SNPs located outside the coding region (introns, 5′ or 3′ boundaries) were numbered relative to their position in the gDNA sequence upstream (“−”) or downstream (“+”) the translational start site (“ATG”, bp = 1).
SNPs located in the coding region were numbered relative to the translational start site on the mRNA sequence.

TABLE 3
Multivariate Analyses of the Association of Donor's TLR
Polymorphisms with the Risk of Invasive Aspergillosis.

| Cohort Study | | | |
|---|---|---|---|
| IA | No IA | HR (95% CI) | P |

SNPs^{§}

| | | | | | |
|---|---|---|---|---|---|
| TLR2 | T/T | 3 (10) | 72 (26) | 1.00 | |
| 597 | T/C | 19 (61) | 148 (53) | 5.85 (1.36-25.16) | 0.02 |
| | C/C | 9 (29) | 59 (21) | 6.65 (1.43-30.89) | 0.02 |
| | T/C or C/C | 28 (90) | 207 (74) | 6.08 (1.44-25.60) | 0.01 |
| TLR3 | C/C | 21 (72) | 140 (50) | 1.00 | |
| 1234 | C/T | 6 (21) | 113 (41) | 0.46 (0.19-1.14) | 0.09 |
| | T/T | 2 (7) | 25 (9) | 0.44 (0.10-1.88) | 0.27 |
| | C/T or T/T | 8 (28) | 138 (50) | 0.46 (0.20-1.03) | 0.06 |
| TLR4 | A/A | 4 (20) | 110 (50) | 1.00 | |
| −2604 | A/G | 12 (60) | 83 (38) | 4.18 (1.33-13.16) | 0.01 |
| | G/G | 4 (20) | 26 (12) | 4.34 (1.08-17.45) | 0.04 |
| | A/G or G/G | 16 (80) | 109 (50) | 4.22 (1.40-12.74) | 0.01 |

6-loci TLR4
Haplotypes

| | | | | | |
|---|---|---|---|---|---|
| H5 | 0/0 | 13 (65) | 187 (84) | 1.00 | |
| | 0/1 | 4 (20) | 32 (14) | 2.14 (0.69-6.66) | 0.19 |
| | 1/1 | 3 (15) | 3 (1) | 5.19 (1.48-18.28) | 0.01* |
| | 0/1 or 1/1 | 7 (35) | 35 (16) | 2.89 (1.14-7.30) | 0.03 |
| H6^{§} | 0/0 | 15 (75) | 204 (92) | 1.00 | |
| | 0/1 | 5 (25) | 18 (8) | 7.65 (2.41-24.29) | <0.001* |
| | 1/1 | | | | |
| | 0/1 or 1/1 | | | | |
| H8 | 0/0 | 18 (90) | 215 (97) | 1.00 | |
| | 0/1 | 2 (10) | 7 (3) | 3.00 (0.68-13.13) | 0.15 |

2-loci TLR4
Haplotypes

| | | | | | |
|---|---|---|---|---|---|
| S2 | 0/0 | 7 (35) | 166 (75) | 1.00 | |
| | 0/1 | 9 (45) | 46 (21) | 5.53 (2.03-15.11) | <0.001* |
| | 1/1 | 4 (20) | 10 (5) | 7.47 (2.17-25.72) | 0.001* |
| | 0/1 or 1/1 | 13 (65) | 56 (25) | 6.03 (2.38-15.30) | <0.001* |

IA stands for invasive aspergillosis and SNP for single nucleotide polymorphism. Since 20 recipients and 94 donors out of 336 were not Caucasian or had missing ethnicity, the denominators were 242 donors and 316 recipients in the cohort study, but denominators may vary due to missing genotypes.
Covariates included in initial pairwise models were age groups (20-40, >40, using 0-19 as a reference group), underlying disease group (hematological malignancy 1^{st} remission, hematological malignancy non 1^{st} remission, other underlying diseases, using CML chronic phase as a reference group), CMV positive serostatus in donor and/or recipient (excepted when serostatus was combined with haplotypes), acute and chronic GVHD (both time-dependant, post transplant covariate) and itraconazole prophylaxis (post-transplant covariate). Covariates that were not significant (P > 0.15) and did not modify the effect of S2 by more than 10% when entered in a pairwise model with S2 were not entered in the final models. Covariates included in the final Cox regression models were a positive CMV serostatus in donor and/or recipient and acute GVHD (time-dependant covariate).
TLR4 haplotypes were indicated by the letter H (6 loci haplotypes) and the letter S (2 loci haplotypes). See FIG. 1D for genetic organization of TLR4.
Hazard Ratios and Odd Ratios are indicated for each genotype versus the WT genotype (reference).
P values are indicated for each genotype versus the WT genotype (P Genotypes) and for the presence versus the absence of the minor allele (P Alleles).
*The significance level after adjusting for multiple testing using the Bonferroni method is 0.00277 for SNPs (considering 18 independent tests) and 0.0125 for haplotypes (considering 4 independent tests). See text for significant Bonferroni corrected values.
^{§}H6 was in strong LD with 1063 A/G [R² = 0.96] and 1363 C/T [R² = 0.91].

TABLE 4
Multivariate Analysis of Risk Factors for Non-Relapse Death.

| Variables | HR (95% CI) | P |
|---|---|---|
| CMV+/S2+ | 2.47 (1.10-5.56) | 0.03§ |
| CMV−/S2+ or CMV+/S2− | 1.15 (0.57-2.33) | 0.70 |
| Hem. Malign. 1^{st} remission | 1.49 (0.85-2.62) | 0.16 |
| Hem. Malign. non-1^{st} remission | 0.55 (0.19-1.60) | 0.27 |
| Other Underlying Disease | 0.74 (0.10-5.76) | 0.78 |
| Acute GVHD | 3.89 (2.24-6.75) | <0.001 |
| Chronic GVHD | 2.19 (0.98-4.88) | 0.05 |

Covariates included in initial pairwise models were age groups (20-40, >40, using 0-19 as a reference group), underlying disease group (hematological malignancy 1^{st} remission, hematological malignancy non 1^{st} remission, other underlying diseases, using CML chronic phase as a reference group), acute and chronic GVHD (both time-dependant, post transplant covariate) and itraconazole prophylaxis (post-transplant covariate). Covariates that were not significant (P > 0.15) and did not modify the effect of S2 by more than 10% when entered in a pairwise model with S2 were not entered in the final models. Similar results were obtained when post-transplant covariates (acute and chronic GVHD) were not included in the model, although the effect was higher: for CMV+/S2+, HR = 2.64 [95% CI 1.25-5.55] P = 0.01 and for CMV−/S2+ or CMV+/S2− (HR = 1.89 [95% CI 1.02-3.51] P = 0.04).
Invasive Aspergillosis (IA, time-dependant variable) was strongly associated with death (HR = 20.12 [11.17-36.23], P < 0.001). Association between CMV+/S2+ and death disappeared when IA was entered in the model (P = 0.57), suggesting that the effect of CMV+/S2+ on death was mainly mediated through IA (IA and CMV+/S2+ being not independent risk factors).

TABLE 5
Univariate Analysis of the Association of TLR Haplotypes in
Recipients and Donors with Cumulative Incidence of Invasive aspergillosis.

| | | Cumulative Incidence of IA (30 months, %) | | | | | |
|---|---|---|---|---|---|---|---|
| | | Patient (N = 316) | | | Donor (N = 242) | | |
| | | Haplotype | Haplotype | | Haplotype | Haplotype | |
| Gene | | Absent | Present | P | Absent | Present | P |
| TLR2 | 1 TGTTG | (12) | (8) | 0.17 | (9) | (8) | 0.93 |
| | 2 AACTG | (8) | (12) | 0.15 | (7) | (10) | 0.52 |
| | 3 AATTG | (10) | (10) | 0.76 | (7) | (10) | 0.43 |
| | 4 TACTG | (10) | (8) | 0.60 | (10) | (2) | 0.08 |
| | 5 AACCG | (9) | (16) | 0.23 | (8) | (10) | 0.68 |
| | 6 TATTG | (10) | (7) | 0.56 | (9) | (5) | 0.64 |
| TLR3 | 1 ATGC | (8) | (12) | 0.21 | (8) | (9) | 0.71 |
| | 2 AACC | (9) | (11) | 0.55 | (8) | (9) | 0.61 |
| | 3 ATGT | (11) | (6) | 0.16 | (9) | (8) | 0.83 |
| | 4 TTGC | (10) | (9) | 0.79 | (10) | (4) | 0.17 |
| | 5 AAGT | (11) | (4) | 0.12 | (8) | (10) | 0.71 |
| | 6 AAGC | (9) | (17) | 0.15 | (8) | (7) | 0.84 |
| TLR4 | H1 | (10) | (10) | 0.78 | (11) | (6) | 0.19 |
| | GATACGG | | | | | | |
| | H2 | (10) | (9) | 0.97 | (9) | (8) | 0.86 |
| | AATACGG | | | | | | |
| | H3 | (10) | (11) | 0.48 | (10) | (4) | 0.16 |
| | AATACCG | | | | | | |
| | H4 | (10) | (11) | 0.86 | (9) | (5) | 0.30 |
| | AGTACGC | | | | | | |
| | H5 | (10) | (8) | 0.59 | (7) | (17) | 0.04 |
| | AGCACGG | | | | | | |
| | H6 | (10) | (10) | 0.90 | (7) | (22) | 0.01 |
| | AGTGTGG | | | | | | |
| | H7 | (10) | (11) | 0.81 | (9) | | 0.29 |
| | AGCACGC | | | | | | |
| | H8 | (10) | (13) | 0.54 | (8) | (22) | 0.07 |
| | AGTACGG | | | | | | |
| | S1 -A---- | (6) | (10) | 0.37 | (13) | (8) | 0.40 |
| | G | | | | | | |
| | S2 -G---- | (10) | (10) | 0.93 | (4) | (19) | <0.001 |
| | G | | | | | | |
| | S3 -G---- | (9) | (11) | 0.82 | (10) | (5) | 0.17 |
| | C | | | | | | |
| TLR9 | 1 TTGA | (9) | (10) | 0.81 | (6) | (9) | 0.40 |
| | 2 CTAG | (10) | (10) | 0.98 | (10) | (7) | 0.35 |
| | 3 TCAG | (11) | (6) | 0.26 | (7) | (12) | 0.13 |

IA stands for invasive aspergillosis, Abs. for haplotype absent, Pres. for haplotype present. Since 20 out of 336 patients and 94 out of 336 donors were not Caucasian or had missing ethnicity, the denominators in the cohort study were 316 patients and 242 donors.
P is the P value for univariate Cox regression model.

TABLE 6
Multivariate Analysis of the Association of Donor's TLR4
Polymorphisms with the Risk of Invasive Aspergillosis.

| Cohort Study | | | |
|---|---|---|---|
| IA N = 20 | No IA N = 222 | HR (95% CI) | P |

SNPs

| | | | | | |
|---|---|---|---|---|---|
| −3612 | A/A | 13 (65) | 105 (49) | 1.00 | |
| | A/G | 6 (30) | 93 (44) | 0.57 (0.22-1.51) | 0.26 |
| | G/G | 1 (5) | 15 (7) | 0.62 (0.08-4.78) | 0.65 |
| | A/G or G/G | 7 (35) | 108 (51) | 0.58 (0.23-1.46) | 0.25 |
| −2604 | A/A | 4 (20) | 110 (50) | 1.00 | |
| | A/G | 12 (60) | 83 (38) | 4.18 (1.33-13.16) | 0.01 |
| | G/G | 4 (20) | 26 (12) | 4.34 (1.08-17.45) | 0.04 |
| | A/G or G/G | 16 (80) | 109 (50) | 4.22 (1.40-12.74) | 0.01 |
| −1607 | T/T | 12 (67) | 166 (77) | 1.00 | |
| | T/C | 3 (17) | 45 (21) | 0.93 (0.27-3.26) | 0.92 |
| | C/C | 3 (17) | 4 (2) | 3.89 (1.12-13.56) | 0.03 |
| | T/C or C/C | 6 (33) | 49 (23) | 1.51 (0.58-3.93) | 0.40 |
| 1063 | A/A | 15 (75) | 204 (92) | 1.00 | |
| | A/G | 5 (25) | 18 (8) | 7.65 (2.41-24.29) | <0.001 |
| +11381 | G/G | 17 (85) | 153 (69) | 1.00 | |
| | G/C | 2 (10) | 63 (28) | 0.29 (0.07-1.28) | 0.10 |
| | C/C | 1 (5) | 6 (3) | 1.20 (0.16-9.07) | 0.86 |
| | G/C or C/C | 3 (15) | 69 (31) | 0.39 (0.11-1.35) | 0.14 |
| +12186 | G/G | 17 (85) | 157 (71) | 1.00 | |
| | G/C | 3 (15) | 57 (26) | 0.46 (0.13-1.57) | 0.21 |
| | C/C | | 7 (3) | | |
| | G/C or C/C | 3 (15) | 64 (29) | 0.43 (0.13-1.46) | 0.18 |
| H1 | 0/0 | 13 (65) | 110 (50) | 1.00 | |
| | 0/1 | 6 (30) | 97 (44) | 0.53 (0.20-1.41) | 0.20 |
| | 1/1 | 1 (5) | 15 (7) | 0.60 (0.08-4.63) | 0.63 |
| | 0/1 or 1/1 | 7 (35) | 112 (50) | 0.54 (0.22-1.36) | 0.19 |
| H2 | 0/0 | 12 (60) | 126 (57) | 1.00 | |
| | 0/1 | 8 (40) | 86 (39) | 0.91 (0.37-2.22) | 0.83 |
| | 1/1 | | 10 (5) | | |
| | 0/1 or 1/1 | 8 (40) | 96 (43) | 0.81 (0.33-2.00) | 0.65 |
| H3 | 0/0 | 17 (85) | 154 (69) | 1.00 | |
| | 0/1 | 2 (10) | 63 (28) | 0.31 (0.07-1.37) | 0.12 |
| | 1/1 | 1 (5) | 5 (2) | 1.29 (0.17-9.81) | 0.80 |
| | 0/1 or 1/1 | 3 (15) | 68 (31) | 0.42 (0.12-1.44) | 0.17 |
| H4 | 0/0 | 17 (85) | 168 (76) | 1.00 | |
| | 0/1 | 3 (15) | 51 (23) | 0.56 (0.16-1.91) | 0.35 |
| | 1/1 | | 3 (1) | | |
| | 0/1 or 1/1 | 3 (15) | 54 (24) | 0.55 (0.16-1.88) | 0.34 |
| H5 | 0/0 | 13 (65) | 187 (84) | 1.00 | |
| | 0/1 | 4 (20) | 32 (14) | 2.14 (0.69-6.66) | 0.19 |
| | 1/1 | 3 (15) | 3 (1) | 5.19 (1.48-18.28) | 0.01 |
| | 0/1 or 1/1 | 7 (35) | 35 (16) | 2.89 (1.14-7.30) | 0.03 |
| H6 | 0/0 | 15 (75) | 204 (92) | 1.00 | |
| | 0/1 | 5 (25) | 18 (8) | 7.65 (2.41-24.29) | <0.001 |
| | 1/1 | | | | |
| | 0/1 or 1/1 | | | | |
| H7 | 0/0 | 20 (100) | 208 (94) | | |
| | 0/1 | | 14 (6) | | |
| H8 | 0/0 | 18 (90) | 215 (97) | 1.00 | |
| | 0/1 | 2 (10) | 7 (3) | 3.00 (0.68-13.13) | 0.15 |

2-loci
Haplotypes

| | | | | | |
|---|---|---|---|---|---|
| S1 | 0/0 | 4 (20) | 27 (12) | 1.00 | |
| | 0/1 | 12 (60) | 83 (37) | 0.98 (0.31-3.11) | 0.97 |
| | 1/1 | 4 (20) | 112 (50) | 0.23 (0.06-0.94) | 0.04 |
| | 0/1 or 1/1 | 16 (80) | 195 (88) | 0.54 (0.18-1.62) | 0.27 |
| S2 | 0/0 | 7 (35) | 166 (75) | 1.00 | |
| | 0/1 | 9 (45) | 46 (21) | 5.53 (2.03-15.11) | <0.001 |
| | 1/1 | 4 (20) | 10 (5) | 7.47 (2.17-25.72) | 0.001 |
| | 0/1 or 1/1 | 13 (65) | 56 (25) | 6.03 (2.38-15.30) | <0.001 |
| S3 | 0/0 | 17 (85) | 158 (71) | 1.00 | |
| | 0/1 | 3 (15) | 58 (26) | 0.46 (0.13-1.56) | 0.21 |
| | 1/1 | | 6 (3) | | |
| | 0/1 or 1/1 | 3 (15) | 64 (29) | 0.43 (0.13-1.46) | 0.18 |

IA stands for invasive aspergillosis and SNP for single nucleotide polymorphism. Since 94 out of 336 donors were not Caucasian or had missing ethnicity, the denominators in the cohort study 242 donors, but denominators may vary due to missing genotypes.
Covariates included in the Final Cox regression models were a positive CMV serostatus in donor and/or recipient and acute GVHD (time-dependant covariate).
TLR4 haplotypes were indicated by the letter H (6 loci haplotypes) and the letter S (2 loci haplotypes). See FIG. 1D for genetic organization of TLR4.
Hazard Ratios are indicated for each genotype versus the WT genotype (reference). P values are indicated for each genotype versus the WT genotype (P Genotypes) and for the presence versus the absence of the minor allele (P Alleles).
*The significance level after adjusting for multiple testing using the Bonferroni method in the cohort study is 0.00277 for SNPs (considering 18 independent tests) and 0.0125 for haplotypes (considering 4 independent tests).
§ H6 was in LD with SNPs 1063 A/G [R² = 0.96] and 1363 C/T [R² = 0.91].

TABLE 7
Individualized Estimate of Genetic Risk.
All possible haplotypes given unphased
genotypes at two SNP loci are listed below:

| | | | | | | Risk |
|---|---|---|---|---|---|---|
| Two SNP 1 | | | | | Relative | Probability |
| Genotypes | | Haplo- | | Relative | Risk | at 36 |
| 2604 | 12186 | types | Frequency | Risk | (Dominant) | months (%) |
| A/A | G/G | AG/AG | 0.431 | 1 | 1 | 3.2 |
| | C/G | AC/AG | 0.003 | 1 | 1 | 3.2 |
| | C/C | AC/AC | 0.000 | 1 | 1 | 3.2 |
| A/G | G/G | AG/GG | 0.119 | 5.53 | 6.03 | 19.3 |
| | C/G | AC/GG | 0.105 | 1.03 | 1.03 | 3.3 |
| | | or | | | | |
| | | AG/GC | | | | |
| | C/C | AC/GC | 0.001 | 1 | 1 | 3.2 |
| G/G | G/G | GG/GG | 0.033 | 7.47 | 6.03 | 19.3 |
| | C/G | GC/GG | 0.029 | 5.53 | 6.03 | 19.3 |
| | C/C | GC/GC | 0.025 | 1 | 1 | 3.2 |

For those individuals with identifiable haplotypes, their genetic risks are estimated by
Pr(D = 1|G₁, G₂) = Pr(D = 1|H, \dot{H}) = Pr(D = 1|Reference Diplotype)RR(H, \dot{H}),
where Pr(D = 1|G₁,G₂) quantifies the probability of being infected in 36 months, the
reference diplotype (RD) includes all diplotypes of not including “GG” haplotype, and
RR is a relative risk of a diplotype (H, \dot{H}) with respect to the reference diplotype.
For individuals with ambiguous haplotypes (i.e., double heterozygotes), their genetic risks are estimated by
$\begin{matrix}Pr ( D = 1 \vert A \text{/} G , G \text{/} C ) = Pr ( D = 1 \vert RD ) w ( AG , GC ) + \\ Pr ( D = 1 \vert RD ) w ( AC , GG ) \\ = Pr ( D = 1 \vert RD ) [ RR ( AG , GC ) w ( AG , GC ) + \\ RR ( AC , GG ) w ( AC , GG ) ]\end{matrix}$ where the weight is the posterial probability of diplotype given genotype:
w(AG, GC) = f_{AG}f_{GC}/(f_{AG}f_{GC} + f_{AC}f_{GG}) and w(AC, GG) = f_{AC}f_{GG}/(f_{AG}f_{GC} + f_{AC}f_{GG}).
Given estimated haplotype frequencies, the posterial probability of observing diplotype AC/GG, for the double heterozygotes, equals 0.007.

TABLE 8
Characteristics of Transplant Recipients in The Discovery and
Validation Studies.

| | Discovery Study | | | Validation Study | | |
|---|---|---|---|---|---|---|
| | | No IA | | | No IA | |
| | IA (%) | (%) | | IA (%) | (%) | |
| A. Patients Characteristics | N = 33 | N = 303 | P‡ | N = 103 | N = 263 | P‡ |
| Donor/Recipient Ethnicity | | | | | | |
| Caucasian/Caucasian | 55 | 67 | | 98 | 99 | |
| Caucasian/Other* | 39 | 27 | | | | |
| Other*/Caucasian | 6 | 6 | | 2 | 1 | |
| Age groups | | | | | | |
| <40 | 58 | 54 | | 34 | 34 | †† |
| >=40 | 42 | 46 | 0.84 | 66 | 66 | †† |
| Donor/Recipient sex | | | | | | |
| Male/Male | 36 | 40 | | 43 | 35 | |
| Male/Female | 27 | 23 | 0.57 | 28 | 25 | 0.65 |
| Female/Male | 21 | 19 | 0.63 | 15 | 25 | 0.05 |
| Female/Female | 15 | 17 | 0.92 | 15 | 16 | 0.51 |
| Transplantation type | | | | | | |
| Matched related | | | | 52 | 57 | †† |
| Mismatch related | | | | 16 | 19 | †† |
| Matched unrelated | 58 | 65 | | 20 | 15 | †† |
| Mismatch unrelated | 42 | 35 | 0.31 | 13 | 9 | †† |
| Underlying Disease | | | | | | |
| CML - chronic phase | 36 | 40 | | 21 | 30 | |
| Hematologic malignancy - 1st remission | 52 | 45 | 0.32 | 61 | 57 | 0.03 |
| Hematologic malignancy - non-1st remission | 9 | 13 | 0.73 | 18 | 13 | 0.03 |
| Other** | 3 | 2 | 0.61 | | | |
| Myeloablative with Total Body Irradiation | 82 | 86 | | 64 | 56 | |
| (TBI) | | | | | | |
| Myeloablative without TBI | 18 | 14 | 0.55 | 36 | 44 | 0.35 |
| Graft source | | | | | | |
| Bone marrow cells§ | 94 | 94 | | 85 | 87 | |
| Peripheral blood cells | 6 | 6 | 0.97 | 15 | 13 | 0.26 |
| Donor/Recipient CMV serostatus | | | | | | |
| D−/R− | 24 | 46 | | 25 | 37 | |
| D−/R+, D+R+ or D+R− | 76 | 54 | 0.01 | 75 | 63 | 0.06 |
| Anti-GVH prophylaxis | | | | | | |
| Containing anti-T cells therapy | 9 | 8 | 0.92 | 12 | 10 | 0.46 |
| Graft versus Host Disease | | | | | | |
| Acute (grade III-IV)- Cum. Incidence - day | 96 | 35 | <0.001 | 46 | 25 | <0.001 |
| 80‡‡ | | | | | | |
| Chronic Extensive- Cum. Incidence - day | 57 | 68 | 0.04 | 17 | 13 | 0.12 |
| 300‡‡ | | | | | | |
| CMV disease | | | | | | |
| Cumulative Incidence - day 150‡‡ | 12 | 4 | 0.08 | 8 | 2 | 0.01 |
| B. Invasive aspergillosis | N = 33 | (%) | | N = 103 | (%) | |
| Diagnosis | | | | | | |
| Proven | 16 | (48) | | 61 | (59) | |
| Probable | 17 | (52) | | 42 | (41) | |
| Disseminated | 7 | (21) | | 21 | (20) | |
| Sinus | 3 | (9) | | 5 | (5) | |
| Other§§ | 3 | (9) | | | | |
| Microbiological documentation | | | | | | |
| Aspergillus fumigatus | 22 | (67) | | 56 | (54) | |
| Aspergillus niger | 3 | (9) | | 1 | (1) | |
| Aspergillus flavus | 1 | (3) | | 4 | (4) | |
| Aspergillus species | 3 | (9) | | 3 | (3) | |
| Mixed infection with Aspergillus species | 2 | (6) | | 14 | (14) | |
| Culture Negative | 2 | (6) | | 25 | (24) | |
| Median time from transplantation (range)† | 100 | (11-732) | | 68 | (4-1384) | |

*Donor ethnicity was missing in 94 cases.
**Aplastic anemia (n = 5), paroxysmal nocturnal hemoglobinuria (n = 1) and idiopathic thrombocytopenia (n = 1).
§One patient with IA in the cohort study was transplanted with cord blood cells.
§§Central nervous system (n = 2); mouth (n = 1)
‡P is the P value for univariate Cox regression models.
‡‡In the case-control study, numbers indicate the percentage of patients with acute or chronic GVHD and CMV disease before the occurrence of invasive aspergillosis in cases, or during the corresponding period in matched controls.
†Median time to IA was 71 days in recipients of an unrelated donor and 68 days in recipient of a related donor.
†† Controls in the case-control study were matched for age, type of transplantation and year of transplantation.

TABLE 9
Univariate Analysis of the Association of Donor TLR4 Polymorphisms
with Cumulative Incidence of Invasive Aspergillosis in The Discovery Study.

| | Cumulative | | | Cumulative | | |
|---|---|---|---|---|---|---|
| | Incidence of IA | | | Incidence of IA | | |
| | (6 months, %) | | | (36 months, %) | | |
| | N = 242 | | | N = 242 | | |
| | Allele | Allele | P | Allele | Allele | P |
| Polymorphism (amino acid change) | Absent | Present | (Log rank) | Absent | Present | (Log rank) |
| Donor TLR4 −3612 A/G (—) | 8 | 6 | 0.62 | 11 | 6 | 0.18 |
| Donor TLR4 −2604 A/G (—) | 4 | 10 | 0.06 | 4 | 13 | 0.01 |
| Donor TLR4 −1607 T/C (—) | 7 | 4 | 0.41 | 7 | 11 | 0.34 |
| Donor TLR4 1363 C/T (T399I) | 5 | 22 | 0.002§ | 7 | 22 | 0.01 |
| Donor TLR4 +11381 G/C (—) | 8 | 4 | 0.34 | 10 | 4 | 0.14 |
| Donor TLR4 +12186 G/C (—) | 8 | 5 | 0.41 | 10 | 5 | 0.20 |
| Donor TLR4 S1 | 3 | 7 | 0.42 | 13 | 8 | 0.35 |
| Donor TLR4 S2† | 8 | 4 | 0.39 | 10 | 4 | 0.18 |
| Donor TLR4 S3 | 6 | 9 | 0.36 | 6 | 17 | 0.01§ |
| Donor TLR4 S4†† | 5 | 22 | 0.002§ | 7 | 22 | 0.01§ |

Numbers indicate cumulative incidence of invasive aspergillosis (IA). Since 94 out of 336 donors were not Caucasian or had missing ethnicity, the denominators in the cohort study was 242 donors.
Simplified TLR4 haplotypes were indicated by the letter S (3 loci haplotypes). See FIG. 1A for genetic organization of TLR4.
†S2 contained individuals carrying the minor alleles of SNPs +12186 G/C
††S4 contained individuals carrying the minor alleles of SNPs 1063 A/G and 1363 C/T (both in strong LD, R² = 0.96)
§The significance level after adjusting for multiple testing using the Bonferroni method in the discovery study is 0.0028 for SNPs (considering 18 independent tests) and 0.0125 for haplotypes (considering 4 independent tests).

TABLE 10
Multivariate Analyses of the Association of Donor's TLR4 Polymorphisms
with the Risk of Invasive

| | Discovery Study | | Validation Study | | | | | |
|---|---|---|---|---|---|---|---|---|
| | Unrelated* | | All** | | Unrelated only* | | Related only** | |
| | N = 242 | | N = 366 | | N = 97, §§ | | N = 269, §§ | |
| | Adj. HR (95% CI) | P | Adj. OR (95% CI) | P | Adj. OR (95% CI) | P | Adj. OR (95% CI) | P |
| Model using SNPs | | | | | | | | |
| (amino acid change) | | | | | | | | |
| Donor TLR4 −2604 | 3.22 (1.02-10.16) | 0.05 | — | — | — | — | — | — |
| A/G (—) | | | | | | | | |
| Donor TLR4 1363 | 4.96 (1.52-16.24) | 0.008 | 2.63 (1.19-5.84) | 0.02 | 4.57 (1.00-21.01) | 0.05 | 2.48 (0.95-6.45) | 0.06 |
| C/T(T399I) | | | | | | | | |
| Donor TLR4 | — | — | 2.00 (1.14-3.49) | 0.02 | — | — | 2.18 (1.13-4.21) | 0.02 |
| +12186 G/C (—) | | | | | | | | |
| Model using | | | | | | | | |
| simplified | | | | | | | | |
| haplotypes | | | | | | | | |
| Donor TLR4 S1 | Ref. | | Ref. | | Ref. | | Ref. | |
| Donor TLR4 S2† | 0.65 (0.19-2.23) | 0.49 | 1.74 (1.04-2.93) | 0.04 | 1.13 (0.45-2.84) | 0.79 | 1.86 (1.01-3.44) | 0.05 |
| Donor TLR4 S3 | 2.20 (1.14-4.25) | 0.02 | 0.64 (0.30-1.34) | 0.23 | 1.32 (0.41-4.17) | 0.64 | 0.42 (0.15-1.15) | 0.09 |
| Donor TLR4 S4†† | 6.16 (1.97-19.26) | 0.002§ | 2.49 (1.15-5.41) | 0.02 | 5.00 (1.04-24.01) | 0.04 | 2.29 (0.93-5.68) | 0.07 |

Results are for multivariate Cox regression models in the discovery study, and conditional logistic regressions in the validation study. Hazard Ratios and Odd Ratios are indicated for the presence versus the absence of the minor allele.
*adjusted for CMV positive serostatus in donor and/or recipient and acute GVHD (as a time-dependant covariate in the discovery study, and during the time from transplantation to invasive aspergillosis in cases from the case-control study, and corresponding time in matched controls).
**adjusted for underlying disease groups, antifungal prophylaxis (itraconazole or voriconazole) and donor/recipient sex. The model was also adjusted for acute and chronic GVHD, as well as CMV disease, during the time from transplantation to invasive aspergillosis in cases, and corresponding time in matched controls.
Simplified TLR4 haplotypes were indicated by the letter S (3 loci haplotypes). See FIG. 1A for genetic organization of TLR4.
†S2 contained individuals carrying the minor alleles of SNPs +12186 G/C
††S4 contained individuals carrying the minor alleles of SNPs 1063 A/G and 1363 C/T (both in strong LD, R² = 0.96)
§ The significance level after adjusting for multiple testing using the Bonferroni method in the discovery study is 0.0028 for SNPs (considering 18 independent tests) and 0.0125 for haplotypes (considering 4 independent tests).
§§ A subgroup analysis was performed to allow comparison between HCT types in the discovery and validation studies.

TABLE 11
Multivariate Analysis of Pre-transplant Risk Factors for Non-Relapse
Death in the Discovery Study.

| Non-Relapse Death | Adjusted HR (95% CI) | P |
|---|---|---|
| CMV+ and/or S4+ | 1.88 (1.10-3.20) | 0.02 |
| Hem. Malign. 1^{st} remission | 1.60 (0.98-2.61) | 0.06 |
| Hem. Malign. non-1^{st} remission | 0.55 (0.19-1.59) | 0.27 |
| Other Underlying disease | 1.11 (0.26-4.67) | 0.89 |

CMV+/− indicated presence/absence of CMV positive serology in HCT donor and or recipient;
S4+/− indicate presence/absence of the TLR4 S4 haplotype in HCT donor.

1. A method of identifying a subject having a haplotype in a toll-like receptor 4 gene of the subject that is associated with an increased risk of invasive mold infection in a recipient of a transplant from the subject, comprising genotyping the subject for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the subject, wherein the detection of said S4 haplotype identifies the subject as having a haplotype associated with an increased risk of invasive mold infection in a recipient of a transplant from the subject.

2. A method of screening a transplant donor for a haplotype in a toll-like receptor 4 gene of the donor that is associated with an increased risk of invasive mold infection in a recipient of a transplant from the donor, comprising genotyping the donor for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363 S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the donor, wherein the detection of said S4 haplotype identifies the donor as having a haplotype associated with an increased risk of invasive mold infection in a recipient of a transplant from the donor.

3. A method of identifying a subject having a haplotype in a toll-like receptor 4 gene of the subject that is associated with an increased risk of invasive mold infection in a recipient of a transplant from the subject, comprising genotyping the subject for the presence of a haplotype in the toll-like receptor gene of the subject selected from the group consisting of:
a) H5;
b) H6; and
c) H8,
wherein the detection of said haplotype identifies the subject as having a haplotype associated with an increased risk of invasive mold infection in a recipient of a transplant from the subject.

4. A method of screening a transplant donor for a haplotype in a toll-like receptor 4 gene of the donor that is associated with an increased risk of invasive mold infection in a recipient of a transplant from the donor, comprising genotyping the donor for the presence of a haplotype in the toll-like receptor gene of the donor selected from the group consisting of:
a) H5;
b) H6; and
c) H8,
wherein the detection of said haplotype identifies the donor as having a haplotype associated with an increased risk of invasive mold infection in a recipient of a transplant from the donor.

5. A method of identifying a subject having a single nucleotide polymorphism allele or a combination of single nucleotide polymorphism alleles in a toll-like receptor 4 gene of the subject that is associated with an increased risk of invasive mold infection in a recipient of a transplant from the subject, comprising genotyping the subject for the presence of one or more than one single nucleotide polymorphism allele in the toll-like receptor gene of the subject selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
wherein the detection of said single nucleotide polymorphism allele or combination of single nucleotide polymorphism alleles identifies the subject as having a single nucleotide polymorphism allele or a combination of single nucleotide polymorphism alleles associated with an increased risk of invasive mold infection in a recipient of a transplant from the subject.

6. A method of screening a transplant donor for a single nucleotide polymorphism allele or a combination of single nucleotide polymorphism alleles in a toll-like receptor 4 gene of the donor that is associated with an increased risk of invasive mold infection in a recipient of a transplant from the donor, comprising genotyping the donor for the presence of one or more than one single nucleotide polymorphism allele in the toll-like receptor gene of the donor selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
wherein the detection of said single nucleotide polymorphism allele or combination of single nucleotide polymorphism alleles identifies the donor as having a single nucleotide polymorphism allele or a combination of single nucleotide polymorphism alleles associated with an increased risk of invasive mold infection in a recipient of a transplant from the donor.

7. A method of identifying a transplant recipient as having an increased risk of invasive mold infection following transplantation, comprising detecting in the transplant recipient a haplotype of a toll-like receptor 4 gene that is associated with an increased risk of invasive mold infection in a transplant recipient, comprising genotyping the transplant recipient for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the transplant recipient, wherein the detection of said S4 haplotype identifies the transplant recipient as having a haplotype associated with an increased risk of invasive mold infection following transplantation and thereby identifies the transplant recipient as having an increased risk of invasive mold infection following transplantation.

8. A method of identifying a transplant recipient as having an increased risk of invasive mold infection following transplantation, comprising detecting in the transplant recipient a haplotype in a toll-like receptor 4 gene of the transplant recipient that is associated with an increased risk of invasive mold infection in a recipient of a transplant, comprising genotyping the transplant recipient for the presence of a haplotype selected from the group consisting of:
a) H5;
b) H6; and
c) H8,
wherein the detection of said haplotype identifies the transplant recipient as having a haplotype associated with an increased risk of invasive mold infection in a recipient of a transplant, thereby identifying the transplant recipient as having an increased risk of invasive mold infection following transplantation.

9. A method of identifying a transplant recipient as having an increased risk of invasive mold infection following transplantation, comprising detecting in the transplant recipient a single nucleotide polymorphism allele or a combination of single nucleotide polymorphism alleles in a toll-like receptor 4 gene of the transplant recipient that is associated with an increased risk of invasive mold infection in a recipient of a transplant, comprising genotyping the transplant recipient for the presence of one or more than one single nucleotide polymorphism allele in the toll-like receptor gene of the transplant recipient selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
wherein the detection of said single nucleotide polymorphism allele or combination of single nucleotide polymorphism alleles identifies the transplant recipient as having a single nucleotide polymorphism allele or a combination of single nucleotide polymorphism alleles associated with an increased risk of invasive mold infection in a recipient of a transplant, thereby identifying the transplant recipient as having an increased risk of invasive mold infection following transplantation.

10. A method of identifying an immunocompromised or high risk subject as having an increased risk of invasive mold infection, comprising detecting in the subject a haplotype of a toll-like receptor 4 gene that is associated with an increased risk of invasive mold infection, comprising genotyping the subject for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the subject, wherein the detection of said S4 haplotype identifies the subject as having a haplotype associated with an increased risk of invasive mold infection and thereby identifies the subject as having an increased risk of invasive mold infection.

11. A method of identifying a immunocompromised or high risk subject as having an increased risk of invasive mold infection, comprising detecting in the subject a haplotype in a toll-like receptor 4 gene of the subject that is associated with an increased risk of invasive mold infection, comprising genotyping the subject for the presence of a haplotype selected from the group consisting of:
a) H5;
b) H6; and
c) H8,
wherein the detection of said haplotype identifies the subject as having a haplotype associated with an increased risk of invasive mold infection, thereby identifying the subject as having an increased risk of invasive mold infection.

12. A method of identifying an immunocompromised subject as having an increased risk of invasive mold infection, comprising detecting in the subject a single nucleotide polymorphism allele or a combination of single nucleotide polymorphism alleles in a toll-like receptor 4 gene of the subject that is associated with an increased risk of invasive mold infection, comprising genotyping the subject for the presence of one or more than one single nucleotide polymorphism allele in the toll-like receptor gene of the subject selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
wherein the detection of said single nucleotide polymorphism allele or combination of single nucleotide polymorphism alleles identifies the subject as having a single nucleotide polymorphism allele or a combination of single nucleotide polymorphism alleles associated with an increased risk of invasive mold, thereby identifying the subject as having an increased risk of invasive mold infection.

13. The method of claim 1, wherein the invasive mold infection is caused by a fungus selected from the group consisting of Aspergillus species, Fusarium species, Mucor species, Rhizopus species and any combination thereof.

14. The method of claim 10, wherein the immunocompromised subject is selected from the group consisting of:
a) a transplant recipient;
b) a cancer patient;
c) a cancer patient undergoing chemotherapy and/or radiation therapy;
d) a critically ill patient;
e) a patient that has an immunosuppressing condition and/or disorder and/or disease;
f) a subject taking immunosuppressive medication;
g) a subject with an immunodeficiency due to a genetic defect;
h) a high risk subject; and
h) any combination thereof.

15. A method of identifying a subject, which can be a transplant donor, a transplant recipient, an immunocompromised subject and/or a high risk subject, as having an increased risk of invasive mold infection, comprising genotyping the subject for the presence of a G allele of the single nucleotide polymorphism rs4986790 (1063) and a T allele of the single nucleotide polymorphism rs4986791 (1363), wherein the detection of said alleles identifies the subject as having an increased risk of invasive mold infection.

16. A method of screening for increased risk of invasive mold infection or increased mortality in an immunocompromised subject, wherein the presence of a haplotype in the toll-like receptor 4 (TLR4) gene of the subject selected from the group consisting of:
a) an S4 haplotype;
b) an H5 haplotype;
c) an H6 haplotype; and
d) an H8 haplotype,
indicates said subject is at increased risk of an invasive mold infection or increased likelihood of mortality, comprising detecting the presence or absence of said haplotype in a biological sample of said subject.

17. The use of a means of detecting a haplotype of a toll-like receptor 4 gene, wherein said haplotype is selected from the group consisting of:
a) an S4 haplotype;
b) an H5 haplotype;
c) an H6 haplotype; and
d) an H8 haplotype,
in a biological sample of a subject, which can be a transplant donor, a transplant recipient, an immunocompromised subject and/or a high risk subject, in determining if said subject is at increased risk of an invasive mold infection or mortality.

18. A method of identifying a transplant recipient as having an increased risk of invasive mold infection following transplantation, comprising detecting in a donor tissue and/or cell in the transplant recipient a haplotype of a toll-like receptor 4 gene that is associated with an increased risk of invasive mold infection in a transplant recipient, comprising genotyping the donor tissue and/or cell in the transplant recipient for the presence of an S4 haplotype comprising a G allele of the single nucleotide polymorphism rs10759931 (−2604), a G allele of the single nucleotide polymorphism rs7873784 (+12186), a G allele of the single nucleotide polymorphism rs4986790 (1063) and a T allele of the single nucleotide polymorphism rs4986791 (1363) in the toll-like receptor 4 gene of the transplant recipient, wherein the detection of said S4 haplotype in the donor tissue and/or cell in the transplant recipient identifies the transplant recipient as having an increased risk of invasive mold infection following transplantation.

19. A method of identifying a transplant recipient as having an increased risk of invasive mold infection following transplantation, comprising detecting in a donor tissue and/or cell in the transplant recipient a haplotype in a toll-like receptor 4 gene that is associated with an increased risk of invasive mold infection in a recipient of a transplant, comprising genotyping the donor tissue and/or cell in the transplant recipient for the presence of a haplotype selected from the group consisting of:
a) H5;
b) H6; and
c) H8,
wherein the detection of said haplotype in the donor tissue and/or cell identifies the transplant recipient as having an increased risk of invasive mold infection following transplantation.

20. A method of identifying a transplant recipient as having an increased risk of invasive mold infection following transplantation, comprising detecting in a donor tissue and/or cell in the transplant recipient a single nucleotide polymorphism allele or a combination of single nucleotide polymorphism alleles in a toll-like receptor 4 gene that is associated with an increased risk of invasive mold infection in a recipient of a transplant, comprising genotyping the donor tissue and/or cell of the transplant recipient for the presence of one or more than one single nucleotide polymorphism allele in the toll-like receptor gene selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above,
wherein the detection in the donor tissue and/or cell of said single nucleotide polymorphism allele or combination of single nucleotide polymorphism alleles identifies the transplant recipient as having an increased risk of invasive mold infection following transplantation.

21. A method of identifying a transplant recipient as having an increased risk of invasive mold infection following transplantation, comprising genotyping a donor tissue and/or cell in the transplant recipient for the presence of a G allele of the single nucleotide polymorphism rs4986790 (1063) and a T allele of the single nucleotide polymorphism rs4986791 (1363), wherein the detection of said alleles in a donor tissue and/or cell identifies the transplant recipient as having an increased risk of invasive mold infection following transplantation.

22. A kit for genotyping a subject for the presence of an allele or haplotype that is associated with an increased risk of invasive mold infection, comprising one or more reagents for detecting an allele selected from the group consisting of:
a) a G allele of the single nucleotide polymorphism rs10759931 (−2604);
b) a G allele of the single nucleotide polymorphism rs7873784 (+12186);
c) an A allele of the single nucleotide polymorphism rs4986790 (1063);
d) a G allele of the single nucleotide polymorphism rs4986790 (1063);
e) a T allele of the single nucleotide polymorphism rs4986791 (1363);
f) a C allele of the single nucleotide polymorphism rs4986791 (1363);
g) an A allele of the single nucleotide polymorphism rs2770150 (−3612);
h) a C allele of the single nucleotide polymorphism rs10759932 (−1607);
i) a T allele of the single nucleotide polymorphism rs10759932 (−1607);
j) a G allele of the single nucleotide polymorphism rs11536889 (+11381); and
k) any combination of (a)-(j) above.

23. The kit of claim 22, comprising one or more reagents for detecting the alleles of the S4 haplotype.

24. The kit of claim 22, comprising one or more reagents for detecting the alleles of the H5 haplotype.

25. The kit of claim 22, comprising one or more reagents for detecting the alleles of the H6 haplotype.

26. The kit of claim 22, comprising one or more reagents for detecting the alleles of the H8 haplotype.

27. The kit of claim 22, comprising one or more reagents for detecting a G allele of the single nucleotide polymorphism rs4986790 (1063) and a T allele of the single nucleotide polymorphism rs4986791 (1363).
